(19) United States
(12) Patent Application Publication
(10) Pub. No.: US 2022/0403005 A1
(43) Pub. Date: Dec. 22, 2022

(54) COMPOSITIONS AND METHODS FOR MODULATING FACTOR VIII FUNCTION

(71) Applicant: THE CHILDREN'S HOSPITAL OF PHILADELPHIA, Philadelphia, PA (US)

(72) Inventors: Lindsey A. George, Haddonfield, NJ (US); Rodney M. Camire, Sicklerville, NJ (US)

(21) Appl. No.: 17/777,245

(22) PCT Filed: Dec. 7, 2020
(86) PCT No.: PCT/US20/63551

§ 371 Date: May 16, 2022

Related U.S. Application Data
(60) Provisional application No. 62/944,718, filed on Dec. 6, 2019.

(51) Int. Cl. C07K 14/755; C12N 15/79; A61P 7/04

(52) U.S. Cl. CPC C07K 14/755; C12N 15/79; A61P 7/04

(57) ABSTRACT

Factor VIII variants and methods of use thereof are disclosed. In accordance with the present invention, compositions and methods for the modulation of hemostasis in patients in need thereof are provided. More specifically. Factor VIII (FVIII) variants which modulate (e.g., increase) hemostasis are provided. In a particular embodiment, the Factor VIII variant comprises at least one mutation at position 336 and/or 562.

This application claims priority under 35 U.S.C. § 119(e) to U.S. Provisional Patent Application No. 62/944,718, filed Dec. 6, 2019. The foregoing application is incorporated by reference herein.

This invention was made with government support under Grant Number NHLBI K08 HL 146991-01 awarded by the National Institutes of Health. The government has certain rights in the invention.

### FIELD OF THE INVENTION

The present invention relates to the fields of medicine and hematology. More specifically, the invention provides novel Factor VIII variants and methods of using the same to modulate the coagulation cascade in patients in need thereof.

### BACKGROUND OF THE INVENTION

Several publications and patent documents are cited throughout the specification in order to describe the state of the art to which this invention pertains. Each of these citations is incorporated herein by reference as though set forth in full.

Coagulation factor VIII (FVIII) circulates in blood tightly bound to its carrier protein, von Willebrand factor (vWF) (Eaton, et al. (1986) Biochemistry 25(2):505-512; Vehar, et al. (1984) Nature 312(5992):337-342; Lollar, et al. (1988) J. Biol. Chem., 263(21):10451-10455). Proteolytic processing by thrombin liberates FVIII from vWF and produces the active cofactor species (FVIIIa), which is a heterotrimer comprised of an A2-domain weakly associated with the metal ion-stabilized A1/A3-C1-C2 heterodimer (Vehar, et al. (1984) Nature (1984) 312(5992):337-342; Fay, et al. (1992) J. Biol. Chem., 267(19):13246-13250). Factor VIIIa associates with activated FIX (FIXa) on anionic phospholipid surfaces forming the intrinsic Xase enzyme complex, one of two enzymes that activates FX (Eaton, et al. (1986) Biochemistry 25(2):505-512; Hill-Eubanks, et al. (1990) J. Biol. Chem., 265(29):17854-17858; Lenting, et al. (1994) J. Biol. Chem., 269(10):7150-7155; Venkateswarlu, D. (2014) Biochem. Biophys. Res. Comm., 452(3):408-414; Kolkman, et al. (1999) Biochem J., 339(Pt 2):217-221; Fay, et al. (1998) J. Biol. Chem., 273(30):19049-19054; Kolkman, et al. (1999) 274(41):29087-29093; Kolkman, et al. (2000) Biochemistry 39(25):7398-7405). Deficiency or dysfunction of FVIII results in hemophilia A (HA), highlighting the importance of FVIIIa cofactor function. Downregulation of intrinsic Xase function is achieved through inhibition of FIXa by antithrombin and possibly protein S (PS), and FVIIIa inactivation by spontaneous A2-domain dissociation or proteolytic cleavage at Arg336 and Arg562 by activated protein C (APC) (Lollar, et al. (1991) J. Biol. Chem., 266(19):12481-12486; Hultin, et al. (1981) Blood 57(3):476-482; Lollar, et al. (1984) Blood 63(6):1303-1308; Lollar, et al. (1990) J. Biol. Chem., 265(3):1688-1692; Walker, et al. (1987) Arch. Biochem. Biophys., 252(1):322-328; Plautz, et al. (2018) Arterioscler. Thromb. Vasc. Biol., 38(4):816-828; Fay, et al. (1991) J. Biol. Chem., 266(30):20139-20145). Because FVIIIa has such a profound effect (10³-10⁶-fold) on increasing FIXa function, its inactivation is important for regulating intrinsic Xase function (van Dieijen, et al. (1981) J. Biol. Chem., 256(7):3433-3442; Mertens, et al. (1984) Biochem. J., 223(3):599-605).

Following activation by thrombin, FVIIIa loses activity in minutes due to spontaneous A2-domain dissociation (Lollar, et al. (1991) J. Biol. Chem., 266(19):12481-12486; Hultin, et al. (1981) Blood 57(3):476-482; Lollar, et al. (1984) Blood 63(6):1303-1308; Lollar, et al. (1990) J. Biol. Chem., 265(3):1688-1692; Lu, et al. (1996) Blood 87(11):4708-4717; Fay, et al. (1991) J. Biol. Chem., 266(14):8957-8962). The physiologic relevance of this mechanism is exemplified by a number of mild HA mutations that diminish A2 affinity within the FVIIIa heterotrimer (McGinniss, et al. (1993) Genomics 15(2):392-398; Duncan, et al. (1994) Br. J. Haematol., 87(4):846-848; Rudzki, et al. (1996) Br. J. Haematol., 94(2):400-406; Hakeos, et al. (2002) Thromb. Haemost., 88(5):781-787; Pipe, et al. (2001) Blood 97(3):685-691; Pipe, et al. (1999) Blood 93(1):176-183). The presumed importance of A2-domain dissociation in regulating FVIIIa function has been exploited to successfully bioengineer variants with enhanced inter-domain interactions that confer improved hemostatic function (Leong, et al. (2015) Blood 125(2):392-398; Wakabayashi, et al. (2008) Blood 112(7):2761-2769; Gale, et al. (2003) J. Thromb. Haemostasis 1(9):1966-1971; Gale, et al. (2008) J. Biol. Chem., 283(24):16355-16362). Collectively, available biochemical, clinical, and in vivo data support A2-domain dissociation is an important mechanism regulating FVIIIa function. In contrast, previous biochemical studies show that FVIIIa inactivation by APC occurs over hours (Fay, et al. (1991) J. Biol. Chem., 266(30):20139-20145; Lu, et al. (1996) Blood 87(11):4708-4717). The faster rate of A2-dissociation compared to APC cleavage has implicated the former as the predominant mechanism of FVIIIa inactivation (Lollar, et al. (1991) J. Biol. Chem., 266(19):12481-12486; Hultin, et al. (1981) Blood 57(3):476-482; Lollar, et al. (1984) Blood 63(6):1303-1308; Lollar, et al. (1990) J. Biol. Chem., 265(3):1688-1692; Lu, et al. (1996) Blood 87(11):4708-4717; Fay, et al. (1991) J. Biol. Chem., 266(14):8957-8962). Consistent with this understanding, there is no described clinical phenotype associated with altered APC cleavage of FVIII/FVIIIa (Bezemer, et al. (2008) JAMA 299(11):1306-1314; EAHAD F8 Gene Variant Database). This is in contrast to FV, which is similar to FVIII, where APC resistance (FV-Leiden, Arg506G1n) imparts a 50 to 100-fold and 5 to 10-fold increased venous thrombosis risk in the homozygous or heterozygous state, respectively, and is the most common inherited thrombophilia (Bertina, et al. (1994) Nature 369(6475):64-67; Zoller, et al. (1994) Lancet 343(8912):1536-1538; Zoller, et al. (1994) J. Clin. Invest., 94(6):2521-2524; Juul, et al. (2002) Blood 100(1):3-10; Suzuki, et al. (1983) J. Biol. Chem., 258:1914-1920).

As explained above, mutations in Factor VIII (FVIII) can lead to severe bleeding disorders and are associated with hemophilia A. Defective FVIII or a lack of FVIII activity results in an inability to effectively form clots. To date, only 20% of patients with hemophilia A worldwide receive regular treatment with FVIII replacement therapy due its high cost. Typically, the FVIII is plasma-derived or recombinantly produced. Gene therapy for hemophilia A based on AAV vectors is promising, but there is a safety limitation due to aberrant immune responses to the vector. Thus, generating enhanced FVIII molecules would benefit the treatment of hemophilia. Therefore, there is an obvious need for FVIII molecules with improved biological properties.

### SUMMARY OF THE INVENTION

In accordance with the present invention, compositions and methods for the modulation of hemostasis in patients in need thereof are provided. More specifically, Factor VIII (FVIII) variants which modulate (e.g., increase) hemostasis are provided. In a particular embodiment, the Factor VIII variant comprises at least one mutation at position 336 and/or 562. In a particular embodiment, the Arg at position 336 and/or 562 is substituted with Gln. Compositions comprising at least one FVIII variant of the instant invention and at least one pharmaceutically acceptable carrier are also provided. Nucleic acid molecules encoding the FVIII variants of the invention are also disclosed as are methods of use thereof. Another aspect of the invention includes host cells expressing the FVIII variants described herein. Methods for isolating and purifying the FVIII variants are also disclosed.

Pharmaceutical compositions comprising the FVIII variants and/or FVIII variant encoding nucleic acid molecules of the invention in a carrier are also provided. The invention also includes methods for the treatment of a hemostasis related disorder in a patient in need thereof comprising administration of a therapeutically effective amount of the FVIII variant and/or FVIII variant encoding nucleic acid molecules, particularly within a pharmaceutical composition. Such methods have efficacy in the treatment of disorders where a pro-coagulant is needed and include, without limitation, hemophilia, particularly hemophilia A.

### BRIEF DESCRIPTION OF THE DRAWINGS

FIG. 1A provides an amino acid sequence of FVIII (SEQ ID NO: 1). The amino acids at positions 336 and 562 are bolded and underlined. The B domain is also indicated with italics and bolding. The thrombin cleavage site arginines at 372, 740, and 1689 are indicated by italics and underlining. The provided amino acid sequence lacks the 19 amino acid signal peptide at the N-terminus (MQIELSTCFFLCLLRFCFS (SEQ ID NO: 2)). FIG. 1B provides a schematic of the FVIII domain structure with thrombin and APC cleavage sites noted.

FIG. 2A provides an SDS-PAGE analysis of 1.5 μM FVIII-WT and FVIII-QQ before and after a 20-minute incubation with 10 nM thrombin. The gel was stained with Coomassie Blue. SC, single chain; HC, heavy chain; LC, light chain. FIG. 2B provides a representative tracing of thrombin generation in HA human plasma reconstituted with varying concentrations of either FVIII-WT (solid line) or FVIII-QQ (dashed line) initiated with 1 pM FXIa in the presence of 4 μM PCPS, and 7.5 mM CaCl₂. FIG. 2C shows the decline in FVIIIa activity due to A2-domain dissociation determined by intrinsic Xase assay. 5 nM FVIIIa-WT (squares) or FVIIIa-QQ (triangles) was incubated with 100 nM thrombin for 30 seconds, and the residual activity of FVIIIa was assessed over a 15-minute incubation. The data shown are representative of three independent experiments.

FIG. 3A provides a western blot analysis of 10 nM FVIIIWT and FVIII-QQ after a 30-minute incubation with 6 nM APC, 20 μM PCPS and 6 nM hirudin. FVIII fragments were visualized with an anti-A2 antibody (GMA-012). FIG. 3B provides a western blot analysis of 10 nM FVIII-WT, FVIII-QQ, and FVIII-R372Q following a 30-minute incubation with 6 nM APC, 20 μM PCPS and 6 nM hirudin. 30 ng of purified protein was loaded on the gel, and FVIII fragments were visualized with GMA-012. FIG. 3C provides a graph of the inactivation of 10 nM FVIII-WT (solid squares) and FVIII-QQ (solid triangles) by 6 nM APC in the presence of 20 μM PCPS and 6 nM hirudin over time in a purified intrinsic Xase assay compared to inactivation of 10 nM FVIII-WT (open squares) and FVIII-QQ (open triangles) by 6 nM APC with 100 nM PS in the presence of 20 μM PCPS and 6 nM hirudin. Initial velocities of FXa generation throughout the incubation were compared to the 0-minute time point to determine residual FVIII activity. Representative plots of duplicate experiments are plotted. The data were fit to an exponential decay or linear regression (FVIII-QQ with APC only). FIG. 3D provides a western blot analysis of 10 nM FVIII-WT, FVIII-QQ, and FVIII-R372Q, 20 μM PCPS and 6 nM hirudin following a 2 and 10-minute incubation with either 100 nM PS or 6 nM APC or 6 nM APC and 100 nM PS. 20 ng of purified protein was loaded on the gel, and FVIII fragments were visualized with GMA-012. FVIII-R372Q is resistant to cleavage at Arg³⁷². FIG. 3E provides a western blot of a timecourse of the cleavage of WT FVIII and FVIII-QQ by APC.

FIGS. 4A-4D show the effect of APC on FVIII-WT/FVIIIa-WT versus FVIII-QQ/FVIIIa-QQ on thrombin generation in reconstituted HA human and mouse plasma. Thrombin generation was evaluated in the presence of increasing APC concentrations in HA plasma reconstituted with FVIII with 4 μM PCPS and 7.5 mM CaCl₂. FIG. 4A: HA human plasma was either reconstituted with 1 nM FVIII-WT (squares) or FVIII-QQ (triangle) and thrombin generation was initiated with 1 pM FXIa. FIG. 4B: 1.5 nM FVIII was activated with 30 nM thrombin for 30 seconds and quenched with 60 nM hirudin. HA human plasma was reconstituted with 0.2 nM FVIIIa-WT or FVIIIa-QQ. Thrombin generation was initiated with 10 pM FXIa. FIG. 4C: HA mouse plasma was reconstituted with 1 nM FVIII-WT (squares) or FVIII-QQ (triangles) and thrombin generation was initiated with 30 pM FXIa. FIG. 4D: 1.5 nM FVIII was activated with 30 nM thrombin for 30 seconds and quenched with 60 nM hirudin. HA mouse plasma was reconstituted with 0.2 nM FVIIIa-WT or FVIIIa-QQ. Thrombin generation was initiated with 400 pM FXIa. In both panels, residual peak thrombin represents the peak thrombin relative to the 0 nM APC condition. The mean±SEM of four independent experiments are plotted. FIG. 4E shows the effect of sTM on FVIII-WT versus FVIII-QQ on thrombin generation in HA human plasma. Thrombin generation was evaluated in the presence of increasing sTM concentrations in HA human plasma reconstituted with 1 nM FVIII-WT (squares) or FVIII-QQ (triangles) in the presence of 4 μM PCPS, 0.1 pM FXIa, and 7.5 mM CaCl₂. Residual peak thrombin represents the peak thrombin relative to the 0 nM sTM condition. The mean±SEM of four independent experiments are plotted. For 0 nM sTM condition, values for peak thrombin (nM): FVIII-WT: 533.65±4.69, FVIII-QQ: 561.85±6.10; lag time (minutes) FIII-WT: 14.5±1.5, FVIII-QQ: 14.0±1.0.

FIGS. 5A-5D show that FVIII-QQ demonstrates superior in vivo hemostatic function or clot formation compared to FVIII-WT in HA mice. HA mice were infused with PBS (open diamonds) or increasing concentrations of FVIIIWT (squares) or FVIII-QQ (triangles) with or without 10 mg/kg mAb1609 as indicated before undergoing a tail clip injury (FIG. 5A) or a 7.5% FeCl₃ injury (FIG. 5C). WT mice infused with PBS (black circles) serve as hemostatically normal controls. Each point represents a single mouse, and the median and inter-quartile ranges are displayed. The Kruskal-Wallis test was used to determine significance relative to the WT PBS control with P-values ≤0.1 considered significant (p≤0.1=*, p≤0.05=**, p≤0.01=***). Dose-dependent vessel occlusion of FVIII-WT and FVIII-QQ were determined by empirically fitting the tail clip (FIG. 5B) and 7.5% FeCl₃ injury (FIG. 5D) data to a logistic function (solid lines). Points represent median values and error bars are IQR. EC50 and EC80 values were determined from logistic fitting. Dotted line indicates the median value of hemostatically normal controls. n.s., not significant. FIG. 5E shows the half-life study of FVIII-WT and FVIII-QQ in HA mice. FVIII activity was determined at denoted time points after HA mice were injected with 125 IU/kg of FVIII-WT or FVIII-QQ. Each point represents three individual mice, and the mean and standard error of the mean are plotted. Half-life values were calculated by fitting the data to an exponential decay curve.

FIGS. 6A-6B show the effect of APC on FVIII-WT/FVIIIa-WT versus FVIII-QQ/FVIIIa-QQ on thrombin generation in reconstituted HA/FVL murine plasma. Thrombin generation was evaluated in the presence of increasing APC concentrations in FVIII reconstituted HA/FVL murine plasma with 4 μM PCPS, and 7.5 mM CaCl₂). FIG. 6A: HA/FVL plasma was either reconstituted with 1 nM FVIII-WT (squares) or FVIII-QQ (triangles) and thrombin generation was initiated with 30 nM FXIa. FIG. 6B: 10 nM FVIII was activated with thrombin (30 nM) for 30 seconds and quenched with 60 nM hirudin. HA/FVL murine plasma was reconstituted with 0.2 nM FVIIIa-WT or FVIIIa-QQ. Thrombin generation was initiated with 400 pM FXIa. In both panels, residual peak thrombin represents the peak thrombin relative to the 0 nM APC condition. The mean±SEM of four independent experiments are plotted.

FIG. 7 shows enhanced hemostatic effect of FVIII-QQ relative to FVIII-WT is APC dependent. HA/FVL mice were infused with PBS (open diamonds), FVIII-WT (squares), or FVIII-QQ (triangles) at 2 μg/kg with or without 10 mg/kg mAPC anticoagulatant inhibitory antibody (mAb1609) as indicated and then underwent tail clip injury. Each point represents one mouse, and the medians with IQR are presented. The Kruskal-Wallis test was used to determine significance relative to the FVL PBS control with P-values ≤0.1 considered significant (p≤0.1=*, p≤0.05=**, p≤0.01=***). n.s., not significant.

FIG. 8A provides a graph of thrombin generation in HA/FVL plasma with increasing concentrations of APC and WT FVIII, FVIII-QQ, FVIII-R336Q, and FVIII-R562Q. FIG. 8B provides a graph of blood loss in HA/FVL mice after a tail clip assay. A FVL mouse control is also shown. Mice were treated with PBS or WT FVIII, FVIII-QQ, FVIII-R336Q, or FVIII-R562Q.

### DETAILED DESCRIPTION OF THE INVENTION

Hemophilia A (HA) and hemophilia B (HB) are X-linked bleeding disorders due to inheritable deficiencies in either coagulation factor VIII (FVIII) or factor IX (FIX), respectively (Peyvandi, et al., Lancet (2016) 388:187-197; Konkle, et al., Hemophilia A in GeneReviews, Adam, et al., eds., University of Washington (1993)). The bleeding phenotype is generally related to the residual factor activity: people with severe disease (factor activity <1% normal) have frequent spontaneous bleeds; people with moderate disease (factor activity 1%-5% normal) rarely have spontaneous bleeds, but bleed with minor trauma; and people with mild disease (factor activity 5%-40% normal) bleed during invasive procedures or trauma. Given this well-defined relationship between factor activity and bleeding phenotype, HA and HB are attractive targets for protein infusion or gene therapy as small increases in factor levels are expected to have a meaningful clinical impact.

As explained above, Factor VIII is central for coagulation activity and mutations in the FVIII gene result in hemophilia A, the most common form of hemophilia. Herein, specific changes in the amino acid sequence of FVIII are shown to be associated with enhanced protein resistance to proteolytic inactivation. Thus, the instant invention provides rationally designed amino acid residue modifications which provide superior variants.

Full-length FVIII is a large, 280-kDa protein primarily expressed in liver sinusoidal endothelial cells (LSECs), as well as extra-hepatic endothelial cells (Fahs, et al., Blood (2014) 123:3706-3713; Everett, et al., Blood (2014) 123:3697-3705). FVIII predominantly circulates as a heterodimer of a heavy chain and a light chain bound through noncovalent metal-dependent interactions (Lenting, et al., Blood (1998) 92:3983-3996). Factor VIII comprises several domains and is 2332 amino acids in length (mature without signal peptide). Generally, the domains are referred to as A1-A2-B-A3-C1-C2. FVIII is translated as a single-peptide chain (single chain) with the domain structure of A1-α1-A2-α2-B-α3-A3-C1-C2. Proteolytic cleavage of FVIII at R-1313 and/or R-1648 by the trans-Golgi protease furin results in heterodimer formation. The FVIII heavy chain (A1-α1-A2-α2-B) and light chain (α3-A3-C1-C2) remain associated through non-covalent metal-ion-dependent interactions occurring between the A1 and A3 domains. Initially, FVIII is in an inactive form bound to von Willebrand factor (vWF). FVIII is activated by cleavage by thrombin (Factor IIa) and release of the B domain. The activated form of FVIII (FVIIIa) separates from vWF and interacts with coagulation factor Factor IXa-leading to the formation of a blood clot via a coagulation cascade. During coagulation, FVIII single chain or heterodimer is activated to its heterotrimeric cofactor form by cleavage by thrombin at R-372, R-740, and R-1689. A2 remains associated with A1-α1 via non-covalent interactions. Inactivation of FVIIIa occurs via spontaneous A2 dissociation and/or proteolytic cleavage, primarily by activated protein C, at R-336 and R-562.

The B domain comprises 40% of the protein (908 amino acids) and is not required for the protein procoagulant activity (Brinkhous, et al., Proc. Natl. Acad. Sci. (1985) 82:8752-8756). The most common B-domain deleted (BDD) FVIII comprises 14 original amino acid residues (SFSQNPPVLKRHQR (SEQ ID NO: 3)) as a linker (Lind, et al. (1995) Eur. J. Biochem., 232(1):19-27). This BDD FVIII is typically referred to as BDD-SQ or hFVIII-SQ. Short peptide linkers (e.g., 25 or fewer amino acids, 20 or fewer amino acids, 15 or fewer amino acids, or 10 or fewer amino acids) substituted for the B-domain can be used in FVIII variants (Lind, et al. (1995) Eur. J. Biochem., 232(1):19-27; Pittman, et al., Blood (1993) 81:2925-2935; Toole, et al., Proc. Natl. Acad. Sci. (1986) 83:5939-5942). In a particular embodiment, the peptide linker comprises a basic amino acid (e.g., Arg, His, or Lys) at position −1 and −4 to Glu1649. This BDD FVIII form is commonly used to produce recombinant BDD-FVIII (— 4.4 Kb) as well for gene therapy (Berntorp, E., Semin. Hematol. (2001) 38(2 Suppl 4):1-3; Gouw, et al., N. Engl. J. Med. (2013) 368:231-239; Xi, et al., J. Thromb. Haemost. (2013) 11:1655-1662; Recht, et al., Haemophilia (2009) 15:869-880; Sabatino, et al., Mol. Ther. (2011) 19:442-449; Scallan, et al., Blood (2003) 102:2031-2037). As noted above, gene therapy using AAV vectors can only use shortened FVIII molecules such as a BDD-FVIII due to the limited packaging capacity of the AAV (4.7 Kb) and other vector systems (Lind, et al. (1995) Eur. J. Biochem., 232(1):19-27). U.S. Pat. No. 8,816,054, incorporated by reference herein, also provides BDD FVIII molecules with linkers of different lengths and sequences.

FVIIIa is a cofactor for FXIa within the intrinsic Xase complex which functions to generate FXa, leading to the propagation of the coagulation cascade. FVIIIa inactivation is considered to be primarily responsible for the downregulation of intrinsic Xase. FVIIIa inactivation is due to 1) spontaneous A2 dissociation or 2) activated Protein C (APC) proteolytic cleavage (e.g., cleavage of A2 into A2N and A2C). Biochemical and clinical data support the importance of A2 dissociation. Indeed, 90% of FVIIIa activity is lost after 5 minutes in a purified system (Lollar, et al. (1991) J. Biol. Chem., 266:12481-12486). Further, clinical data shows that ⅓ of patients with mild hemophilia have mutations that result in enhanced A2 dissociation. With regard to cleavage, APC cleavage results in the loss of 90% of FVIII activity after 4 hours in a purified system (Lu et al. (1996) Blood 87(11):4708-17). However, unlike alterations in A2 dissociation, no known clinical phenotype is associated with altered APC cleavage.

While available data fails to identify a significant role of APC in regulating FVIIIa function, the lack of a clinical phenotype does not exclude the potential significance of APC-mediated cleavage in FVIIIa inactivation. Further, attempting to ascribe physiologic significance to either FVIII A2-domain dissociation or APC inactivation based on in vitro rates of inactivation alone should be approached with caution. Numerous experimental conditions, many non-physiologic, have been used to study these mechanisms complicating interpretation and perceived significance. Surprisingly, despite decades of FVIII research, the role of APC in FVIIIa regulation in vivo has not been examined.

To study the contribution of APC cleavage in FVIIIa inactivation, Gln missense mutations were introduced at the two known FVIII APC cleavages sites, Arg336 and Arg562, generating a FVIII variant (FVIII-R336Q/R562Q [FVIII-QQ]) resistant to APC cleavage. Consistent with APC having a significant in vivo role in FVIIIa regulation, FVIII-QQ demonstrated superior hemostatic efficacy relative to wild-type FVIII in an APC-dependent manner.

In accordance with the instant invention, novel Factor VIII variants are provided. The instant invention encompasses FVIII variants including FVIIIa variants and FVIII prepeptide variants. For simplicity, the variants are generally described throughout the application in the context of FVIII. However, the invention contemplates and encompasses Factor FVIIIa and FVIII prepeptide molecules as well as Factor VIII domain(s) (e.g., A1 and/or A2 domain) having the same amino acid substitutions and/or linkers as described in FVIII. In a particular embodiment, the FVIII variants of the instant invention are expressed as a single chain molecule or at least almost exclusively as a single chain molecule. In a particular embodiment, the FVIII variants are B-domain deleted (BDD) FVIII (optionally comprising a linker in place of the B-domain). In a particular embodiment, the FVIII variants comprise A1-α1-A2-α2-B-α3-A3-C1-C2. In a particular embodiment, the FVIII variants comprise A1-α1-A2-α2-α3-A3-C1-C2. In a particular embodiment, the FVIII variants comprise A1-α1-A2-α2-A3-C1-C2. In a particular embodiment, the FVIII variants comprise a light chain and a heavy chain (e.g., as a single chain molecule).

As demonstrated herein, the FVIII variants of the instant invention possess greater resistance to APC cleavage than WT FVIII. Moreover, it is also demonstrated herein that the FVIII variants of the instant invention have an unexpectedly superior hemostatic effect compared to WT FVIII. Previously, APC-resistant FVIII would have been expected to have approximately the same in vivo function as WT FVIII because, as explained above, A2 dissociation was considered the predominant mechanism of FVIIIa inactivation. As shown herein, the FVIII variants of the instant invention possess the same activity properties as WT FVIII, but the FVIII variants of the instant invention unexpectedly demonstrated ˜5-fold better in vivo hemostatic function than the wild type protein.

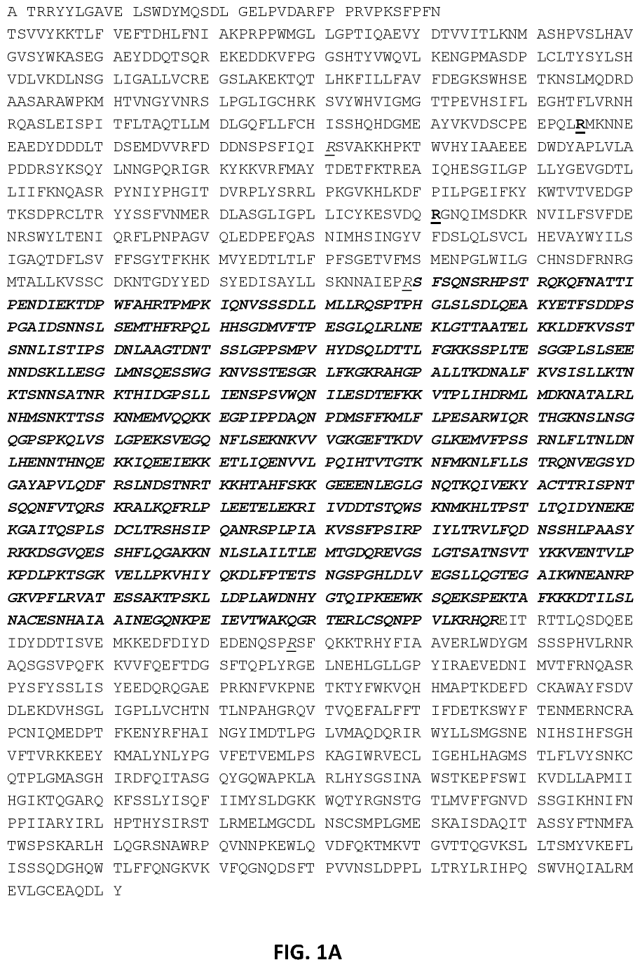

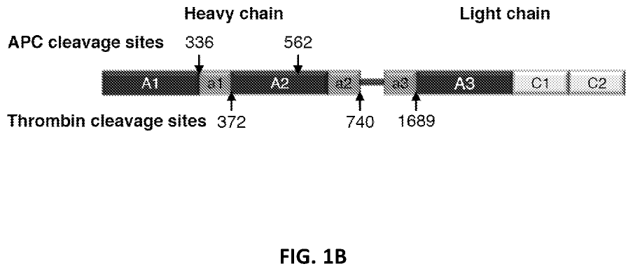

The FVIII variants of the instant invention can be from any mammalian species. In a particular embodiment, the FVIII variant is human. Gene ID: 2157 and GenBank Accession Nos. NM_000132.3 and NP_000123.1 provide examples of the amino acid and nucleotide sequences of wild-type human FVIII (particularly the prepeptide comprising the signal peptide). FIG. 1 provides SEQ ID NO: 1, which is an example of the amino acid sequence of human FVIII. SEQ ID NO: 1 lacks the 19 amino acid signal peptide at its N-terminus (MQIELSTCFFLCLLRFCFS (SEQ ID NO: 2)). Nucleic acid molecules which encode Factor FVIII variants can be readily determined from the provided amino acid sequences as well as the provided GenBank Accession Nos.

In accordance with another aspect of the instant invention, the Factor VIII variants comprise at least one mutation at position 336 and/or 562. As seen herein, these FVIII variants possess greater resistance to cleavage by APC than wild type FVIII. In certain embodiments, the Factor VIII variants comprise a mutation at position 336. In a particular embodiment, the Arg (R) at position 336 is not substituted with Lys (K). In a particular embodiment, the Arg at position 336 is substituted with Asp (D), Glu (E), Asn (N), or Gln (Q). In a particular embodiment, the Arg at position 336 is substituted with Asn (N) or Gln (Q). In a particular embodiment, the Arg at position 336 is substituted with Gln (Q).

In certain embodiments, the Factor VIII variants comprise a mutation at position 562. In a particular embodiment, the Arg (R) at position 562 is not substituted with Lys (K). In a particular embodiment, the Arg at position 562 is substituted with Asp (D), Glu (E), Asn (N), or Gln (Q). In a particular embodiment, the Arg at position 562 is substituted with Asn (N) or Gln (Q). In a particular embodiment, the Arg at position 562 is substituted with Gln (Q).

As stated hereinabove, the FVIII variant of the instant invention may be human. In a particular embodiment, the FVIII variant of the instant invention has at least 75%, 80%, 85%, 90%, 95%, 97%, 99%, or 100% homology (identity) with SEQ ID NO: 1 (or fragment or domain thereof or an activated FVIII fragment thereof), particularly at least 90%, 95%, 97%, 99%, or 100% homology (identity). In a particular embodiment, the FVIII variant comprises an amino acid sequence having at least 75%, 80%, 85%, 90%, 95%, 97%, 99%, or 100% homology (identity), particularly at least 90%, 95%, 97%, 99%, or 100% homology (identity), with amino acids 1-740 of SEQ ID NO: 1 (or fragment or domain thereof or an activated FVIII fragment thereof) and an amino acid sequence having at least 75%, 80%, 85%, 90%, 95%, 97%, 99%, or 100% homology (identity), particularly at least 90%, 95%, 97%, 99%, or 100% homology (identity) with amino acids 1649-2332 or 1690-2332 of SEQ ID NO: 1 (or fragment or domain thereof or an activated FVIII fragment thereof). The homology (identity) percentages above exclude the substitutions at position 336 and/or 562.

The FVIII variants of the instant invention may also be post-translationally modified. The FVIII variants may be post-translationally modified in a cell (particularly a human cell) or in vitro.

In a particular embodiment, the FVIII variants of the invention have increased resistance to cleavage and/or inactivation (e.g., by APC) compared to wild-type FVIII.

Nucleic acid molecules encoding the above FVIII variants (or fragments or domains thereof or activated fragments thereof) are also encompassed by the instant invention. Nucleic acid molecules encoding the variants may be prepared by any method known in the art. The nucleic acid molecules may be maintained in any convenient vector, particularly an expression vector.

Compositions comprising at least one FVIII variant and at least one carrier (e.g., pharmaceutically acceptable carrier) are also encompassed by the instant invention. In a particular embodiment, the FVIII is isolated and/or substantially pure within the composition. Compositions comprising at least one FVIII variant nucleic acid molecule and at least one carrier are also encompassed by the instant invention. Except insofar as any conventional carrier is incompatible with the variant to be administered, its use in the pharmaceutical composition is contemplated. In a particular embodiment, the carrier is a pharmaceutically acceptable carrier for intravenous administration.

### Definitions

Various terms relating to the biological molecules of the present invention are used hereinabove and also throughout the specification and claims.

The phrase “hemostasis related disorder” refers to bleeding disorders such as, without limitation, hemophilia A, hemophilia B, hemophilia A and B patients, hemophilia with inhibitory antibodies, deficiencies in at least one coagulation factor (e.g., Factors VII, VIII, IX, X, XI, V, XII, II, and/or von Willebrand factor, particularly Factor VIII), combined FV/FVIII deficiency, vitamin K epoxide reductase C1 deficiency, gamma-carboxylase deficiency, bleeding associated with trauma or injury, thrombosis, thrombocytopenia, stroke, coagulopathy (hypocoagulability), disseminated intravascular coagulation (DIC), over-anticoagulation associated with heparin, low molecular weight heparin, pentasaccharide, warfarin, or small molecule antithrombotics (e.g., FXa inhibitors); and platelet disorders such as, Bernard Soulier syndrome, Glanzman thromblastemia, and storage pool deficiency. In a particular embodiment, the term “hemostasis related disorder” refers to bleeding disorders characterized by excessive and/or uncontrolled bleeding (e.g., a disorder which can be treated with a procoagulant). In a particular embodiment, the hemostasis related disorder is hemophilia. In a particular embodiment, the hemostasis related disorder is hemophilia A.

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote. With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

With respect to protein, the term “isolated protein” is sometimes used herein. This term may refer to a protein produced by expression of an isolated nucleic acid molecule of the invention. Alternatively, this term may refer to a protein which has been sufficiently separated from other proteins with which it would naturally be associated (e.g., so as to exist in “substantially pure” form). “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “vector” refers to a carrier nucleic acid molecule (e.g., RNA or DNA) into which a nucleic acid sequence can be inserted for introduction into a host cell where it will be replicated. An “expression vector” is a specialized vector that contains a gene or nucleic acid sequence with the necessary regulatory regions (e.g., promoter) needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “substantially pure” refers to a preparation comprising at least 50-60% by weight the compound of interest (e.g., nucleic acid, oligonucleotide, protein, etc.), particularly at least 75% by weight, or at least 90-99% or more by weight of the compound of interest. Purity may be measured by methods appropriate for the compound of interest (e.g. chromatographic methods, agarose or polyacrylamide gel electrophoresis, HPLC analysis, and the like).

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

### Preparation of Variant Encoding Nucleic Acid Molecules and Polypeptides

Nucleic acid molecules encoding the variants of the invention may be prepared by using recombinant DNA technology methods. The availability of nucleotide sequence information enables preparation of isolated nucleic acid molecules of the invention by a variety of means. For example, nucleic acid sequences encoding a variant may be isolated from appropriate biological sources using standard protocols well known in the art.

Nucleic acids of the present invention may be maintained as RNA or DNA in any convenient cloning vector. In a particular embodiment, clones are maintained in a plasmid cloning/expression vector (e.g., pBluescript (Stratagene, La Jolla, Calif.)), which is propagated in a suitable E. coli host cell. Alternatively, the nucleic acids may be maintained in a vector suitable for expression in mammalian cells. In cases where post-translational modification affects variant function, it is preferable to express the molecule in mammalian cells, particularly human cells.

FVIII variant encoding nucleic acid molecules of the invention include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention. Such oligonucleotides are useful as probes for detecting variant expression.

The FVIII variants of the present invention may be prepared in a variety of ways, according to known methods. The protein may be purified from appropriate sources (e.g., transformed bacterial or animal (e.g., mammalian or human) cultured cells or tissues which express FVIII variants), for example, by immunoaffinity purification. The availability of nucleic acid molecules encoding the variants enables production of the variants using in vitro expression methods known in the art. For example, a cDNA or gene may be cloned into an appropriate in vitro transcription vector, such as pSP64 or pSP65 for in vitro transcription, followed by cell-free translation in a suitable cell-free translation system, such as wheat germ or rabbit reticulocyte lysates. In vitro transcription and translation systems are commercially available, e.g., from Promega or Life Technologies.

Alternatively, larger quantities of variant may be produced by expression in a suitable prokaryotic or eukaryotic expression system. For example, part or all of a DNA molecule encoding the FVIII variant may be inserted into a plasmid vector adapted for expression in a bacterial cell, such as E. coli, or a mammalian cell (particularly a human cell) such as CHO or HeLa cells. Alternatively, tagged fusion proteins comprising the variant can be generated. Such variant-tagged fusion proteins are encoded by part or all of a DNA molecule, ligated in the correct codon reading frame to a nucleotide sequence encoding a portion or all of a desired polypeptide tag which is inserted into a plasmid vector adapted for expression in a bacterial cell, such as E. coli or a eukaryotic cell, such as, but not limited to, yeast and mammalian cells, particularly human cells. Vectors such as those described above comprise the regulatory elements necessary for expression of the DNA in the host cell positioned in such a manner as to permit expression of the DNA in the host cell. Such regulatory elements required for expression include, but are not limited to, promoter sequences, transcription initiation sequences, and enhancer sequences.

FVIII variant proteins, produced by gene expression in a recombinant prokaryotic or eukaryotic system (particularly human) may be purified according to methods known in the art. In a particular embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise, without limitation, the FLAG epitope, GST or the hemagglutinin epitope. Such methods are commonly used by skilled practitioners.

FVIII variant proteins, prepared by the aforementioned methods, may be analyzed according to standard procedures. For example, such proteins may be subjected to amino acid sequence analysis, according to known methods.

As discussed above, a convenient way of producing a polypeptide according to the present invention is to express nucleic acid encoding it, by use of the nucleic acid in an expression system. A variety of expression systems of utility for the methods of the present invention are well known to those of skill in the art.

Accordingly, the present invention also encompasses a method of making a polypeptide (as disclosed), the method including expression from nucleic acid encoding the polypeptide (generally nucleic acid). This may conveniently be achieved by culturing a host cell, containing such a vector, under appropriate conditions which cause or allow production of the polypeptide. Polypeptides may also be produced in in vitro systems, such as in reticulocyte lysates.

### Uses of FVIII Variant Proteins and Variant-Encoding Nucleic Acids

FVIII variant proteins and nucleic acids of the instant invention may be used, for example, as therapeutic and/or prophylactic agents which modulate the blood coagulation cascade. The FVIII variant proteins and nucleic acids of the instant invention may be administered in a therapeutically effective amount to modulate (e.g., increase) hemostasis and/or form a clot and/or stop or inhibit bleeding or aberrant bleeding. It is demonstrated herein that the FVIII variants possess superior properties and can provide effective hemostasis.

In a particular embodiment of the present invention, FVIII variants may be administered to a patient via infusion in a biologically compatible carrier, e.g., via intravenous injection. The FVIII variants of the invention may optionally be encapsulated into liposomes or mixed with other phospholipids or micelles to increase stability of the molecule. FVIII variants may be administered alone or in combination with other agents known to modulate hemostasis (e.g., vFW, Factor IX, Factor IXa, etc.). An appropriate composition in which to deliver the FVIII variant may be determined by a medical practitioner upon consideration of a variety of physiological variables, including, but not limited to, the patient's condition and hemodynamic state. A variety of compositions well suited for different applications and routes of administration are well known in the art and are described hereinbelow.

The preparation containing the FVIII variants may contain a physiologically acceptable matrix and is formulated as a pharmaceutical preparation. The preparation can be formulated using substantially known methods, it can be mixed with a buffer containing salts, such as NaCl, CaCl₂, and amino acids, such as glycine and/or lysine, and in a pH range from 6 to 8. Until needed, the purified preparation containing the FVIII variant can be stored in the form of a finished solution or in lyophilized or deep-frozen form. In a particular embodiment, the preparation is stored in lyophilized form and is dissolved into a visually clear solution using an appropriate reconstitution solution. Alternatively, the preparation according to the present invention can also be made available as a liquid preparation or as a liquid that is deep-frozen. The preparation according to the present invention may be especially stable, i.e., it can be allowed to stand in dissolved form for a prolonged time prior to application.

The preparation according to the present invention can be made available as a pharmaceutical preparation with the FVIII variant in the form of a one-component preparation or in combination with other factors in the form of a multi-component preparation.

Prior to processing the purified protein into a pharmaceutical preparation, the purified protein may be subjected to the conventional quality controls and fashioned into a therapeutic form of presentation. In particular, during the recombinant manufacture, the purified preparation may be tested for the absence of cellular nucleic acids as well as nucleic acids that are derived from the expression vector.

Another feature of this invention relates to making available a preparation which contains a FVIII variant with a high stability and structural integrity and which, in particular, is free from inactive FVIII intermediates and/or proteolytic degradation products and and by formulating it into an appropriate preparation.

The pharmaceutical preparation may contain, as an example, dosages of between about 1-1000 μg/kg, about 10-500 μg/kg, about 10-250 μg/kg, or about 10-100 μg/kg. In a particular embodiment, the pharmaceutical protein preparation may comprise a dosage of between 30-100 IU/kg (e.g., as a single daily injection or up to 3 times or more/day). Patients may be treated immediately upon presentation at the clinic with a bleed or prior to the delivery of cut/wound causing a bleed. Alternatively, patients may receive a bolus infusion every one to three, eight, or twelve hours or, if sufficient improvement is observed, a once daily infusion of the FVIII variant described herein.

FVIII variant-encoding nucleic acids may be used for a variety of purposes in accordance with the present invention. In a particular embodiment of the invention, a nucleic acid delivery vehicle (e.g., an expression vector such as a viral vector) for modulating blood coagulation is provided wherein the expression vector comprises a nucleic acid sequence coding for a FVIII variant as described herein. Administration of the FVIII variant-encoding expression vectors to a patient results in the expression of the FVIII variant which serves to alter the coagulation cascade. In accordance with the present invention, a FVIII variant encoding nucleic acid sequence may encode a variant polypeptide as described herein whose expression increases hemostasis. In a particular embodiment, the nucleic acid sequence encodes a human FVIII variant.

Expression vectors comprising FVIII variant nucleic acid sequences may be administered alone, or in combination with other molecules useful for modulating hemostasis. According to the present invention, the expression vectors or combination of therapeutic agents may be administered to the patient alone or in a pharmaceutically acceptable or biologically compatible composition.

In a particular embodiment of the invention, the expression vector comprising nucleic acid sequences encoding the FVIII variant is a viral vector. Viral vectors which may be used in the present invention include, but are not limited to, adenoviral vectors (with or without tissue specific promoters/enhancers), adeno-associated virus (AAV) vectors of any serotype (e.g., AAV-1 to AAV-12, particularly AAV-2, AAV-5, AAV-7, and AAV-8) and hybrid AAV vectors, lentivirus vectors and pseudo-typed lentivirus vectors (e.g., Ebola virus, vesicular stomatitis virus (VSV), and feline immunodeficiency virus (FIV)), herpes simplex virus vectors, vaccinia virus vectors, and retroviral vectors. In a particular embodiment, the vector is an adeno-associated virus (AAV) vector. In a particular embodiment, the vector is a lentiviral vector.

In a particular embodiment of the present invention, methods are provided for the administration of a viral vector comprising nucleic acid sequences encoding a FVIII variant. Adenoviral vectors of utility in the methods of the present invention preferably include at least the essential parts of adenoviral vector DNA. As described herein, expression of a FVIII variant following administration of such an adenoviral vector serves to modulate hemostasis, particularly to enhance the procoagulation activity of the protease.

Recombinant adenoviral vectors have found broad utility for a variety of gene therapy applications. Their utility for such applications is due largely to the high efficiency of in vivo gene transfer achieved in a variety of organ contexts.

Adenoviral particles may be used to advantage as vehicles for adequate gene delivery. Such virions possess a number of desirable features for such applications, including: structural features related to being a double stranded DNA nonenveloped virus and biological features such as a tropism for the human respiratory system and gastrointestinal tract. Moreover, adenoviruses are known to infect a wide variety of cell types in vivo and in vitro by receptor-mediated endocytosis. Attesting to the overall safety of adenoviral vectors, infection with adenovirus leads to a minimal disease state in humans comprising mild flu-like symptoms.

Due to their large size (˜36 kilobases), adenoviral genomes are well suited for use as gene therapy vehicles because they can accommodate the insertion of foreign DNA following the removal of adenoviral genes essential for replication and nonessential regions. Such substitutions render the viral vector impaired with regard to replicative functions and infectivity. Of note, adenoviruses have been used as vectors for gene therapy and for expression of heterologous genes.

It is desirable to introduce a vector that can provide, for example, multiple copies of a desired gene and hence greater amounts of the product of that gene. Improved adenoviral vectors and methods for producing these vectors have been described in detail in a number of references, patents, and patent applications, including: Wright (Hum Gen Ther. (2009) 20:698-706); Mitani and Kubo (Curr Gene Ther. (2002) 2(2):135-44); Olmsted-Davis et al. (Hum Gene Ther. (2002) 13(11):1337-47); Reynolds et al. (Nat Biotechnol. (2001) 19(9):838-42); U.S. Pat. Nos. 5,998,205, 6,228,646, 6,093,699, and 6,100,242; WO 94/17810; and WO 94/23744.

For some applications, an expression construct may further comprise regulatory elements which serve to drive expression in a particular cell or tissue type. Such regulatory elements are known to those of skill in the art. The incorporation of tissue specific regulatory elements in the expression constructs of the present invention provides for at least partial tissue tropism for the expression of the variant or functional fragments thereof. For example, an E1 deleted type 5 adenoviral vector comprising nucleic acid sequences encoding variant under the control of a cytomegalovirus (CMV) promoter may be used to advantage in the methods of the present invention. Hematopoietic or liver specific promoters may also be used.

AAV for recombinant gene expression have been produced in the human embryonic kidney cell line 293 (Wright, Hum Gene Ther (2009) 20:698-706; Graham et al. (1977) J. Gen. Virol. 36:59-72). Briefly, AAV vectors are typically engineered from wild-type AAV, a single-stranded DNA virus that is non-pathogenic. The parent virus is non-pathogenic, the vectors have a broad host range, and they can infect both dividing and non-dividing cells. The vector is typically engineered from the virus by deleting the rep and cap genes and replacing these with the transgene of interest under the control of a specific promoter. For recombinant AAV preparation, the upper size limit of the sequence that can be inserted between the two ITRs is about 4.7 kb. Plasmids expressing a FVIII variant under the control of the CMV promoter/enhancer and a second plasmid supplying adenovirus helper functions along with a third plasmid containing the AAV-2 rep and cap genes may be used to produce AAV-2 vectors, while a plasmid containing either AAV-1, AAV-6, or AAV-8 cap genes and AAV-2 rep gene and ITR's may be used to produce the respective alternate serotype vectors (e.g., Gao et al. (2002) Proc. Natl. Acad. Sci. USA 99:11854-11859; Xiao et al., (1999) J. Virol. 73:3994-4003; Arruda et al., (2004) Blood 103:85-92). AAV vectors may be purified by repeated CsCl density gradient centrifugation and the titer of purified vectors determined by quantitative dot-blot hybridization. In a particular embodiment, vectors may be prepared by the Vector Core at The Children's Hospital of Philadelphia.

Also included in the present invention is a method for modulating hemostasis comprising providing cells of an individual with a nucleic acid delivery vehicle encoding a FVIII variant and allowing the cells to grow under conditions wherein the FVIII variant is expressed.

From the foregoing discussion, it can be seen that FVIII variants and FVIII variant expressing nucleic acid vectors may be used in the treatment of disorders associated with aberrant blood coagulation.

The expression vectors of the present invention may be incorporated into pharmaceutical compositions that may be delivered to a subject, so as to allow production of a biologically active protein (e.g., a FVIII variant) or by inducing expression of the FVIII variant in vivo by gene- and or cell-based therapies or by ex vivo modification/transduction of the patient's or donor's cells. In a particular embodiment of the present invention, pharmaceutical compositions comprising sufficient genetic material to enable a recipient to produce a therapeutically effective amount of a FVIII variant can influence hemostasis in the subject. Alternatively, as discussed above, an effective amount of the FVIII variant may be directly infused into a patient in need thereof. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents (e.g., co-factors) which influence hemostasis.

In particular embodiments, the compositions (e.g., pharmaceutical compositions) of the instant invention also contain a pharmaceutically acceptable carrier. Such carriers include any pharmaceutical agent that does not itself induce an immune response harmful to the individual receiving the composition, and which may be administered without undue toxicity. Pharmaceutically acceptable carriers include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., 18th Edition, Easton, Pa. [1990]).

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, the preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they may be placed in an appropriate container and labeled for treatment. For administration of FVIII variants or FVIII variant encoding vectors, such labeling could include amount, frequency, and method of administration.

Pharmaceutical compositions suitable for use in the invention include compositions wherein the active ingredients are contained in an effective amount to achieve the intended therapeutic purpose. Determining a therapeutically effective dose is well within the capability of a skilled medical practitioner using the techniques and guidance provided in the present invention. Therapeutic doses will depend on, among other factors, the age and general condition of the subject, the severity of the aberrant blood coagulation phenotype, and the strength of the control sequences regulating the expression levels of the variant polypeptide. Thus, a therapeutically effective amount in humans will fall in a relatively broad range that may be determined by a medical practitioner based on the response of an individual patient to vector-based variant treatment.

The FVIII variants, alone or in combination with other agents, may be directly infused into a patient in an appropriate biological/pharmaceutical carrier as described hereinabove. Expression vectors of the present invention comprising nucleic acid sequences encoding variant or functional fragments thereof, may be administered to a patient by a variety of means (see below) to achieve and maintain a prophylactically and/or therapeutically effective level of the variant polypeptide. One of skill in the art could readily determine specific protocols for using the variant encoding expression vectors of the present invention for the therapeutic treatment of a particular patient. Protocols for the generation of adenoviral vectors and administration to patients have been described in U.S. Pat. Nos. 5,998,205; 6,228,646; 6,093,699; and 6,100,242; WO 94/17810 and WO 94/23744, which are incorporated herein by reference in their entirety.

FVIII variants and/or FVIII variant encoding nucleic acids (e.g., adenoviral vectors) of the present invention may be administered to a patient by any means known. Direct delivery of the pharmaceutical compositions in vivo may generally be accomplished via injection using a conventional syringe, although other delivery methods such as convection-enhanced delivery are envisioned (See e.g., U.S. Pat. No. 5,720,720). In this regard, the compositions may be delivered subcutaneously, epidermally, intradermally, intrathecally, intraorbitally, intramucosally, intraperitoneally, intravenously, intraarterially, orally, intrahepatically or intramuscularly. Other modes of administration include oral and pulmonary administration, suppositories, and transdermal applications. A clinician specializing in the treatment of patients with blood coagulation disorders may determine the optimal route for administration of the adenoviral vectors comprising variant nucleic acid sequences based on a number of criteria, including, but not limited to: the condition of the patient and the purpose of the treatment (e.g., enhanced or reduced blood coagulation).

The present invention also encompasses AAV vectors comprising a nucleic acid sequence encoding a FVIII variant. Also provided are lentiviruses or pseudo-typed lentivirus vectors comprising a nucleic acid sequence encoding a FVIII variant. Also encompassed are naked plasmid or expression vectors comprising a nucleic acid sequence encoding a FVIII variant.

The following example is provided to illustrate various embodiments of the present invention. The example is illustrative and is not intended to limit the invention in any way.

### EXAMPLE

### Materials and Methods

### Reagents

The inhibitors benzamidine and 4-amidinophenylmethanesulfonyl fluoride hydrochloride (APMSF) were obtained from Sigma Aldrich (St. Louis, Mo.). Cell culture reagents were from Invitrogen (Waltham, Mass.) except for insulin-transferrin-sodium selenite which was purchased from Roche (Basel, Switzerland). Synthetic phospholipids vesicles (PCPS) were prepared from 75% hen egg L-α-phosphatidylcholine (PC) and 25% porcine brain L-α-Phosphatidylserine (PS) (Avanti Polar Lipids; Alabaster, Ala.) and quantified as described (Pittman, et al. (1993) Blood 81(11):2925-2935). Triniclot reagent (Tcoag) was used to measure automated activated partial thromboplastin time (aPTT). The peptidyl substrate, Spectrozyme® Xa (Sekisui Diagnostics; Burlington, Mass.) was prepared in water and concentration was verified using E342=8279 M⁻¹cm⁻¹ (Lottenberg, et al. (1983) Biochim. Biophys. Acta., 742(3):558-564). The fluorogenic substrate, 0.5 mM Z-Gly-Gly-Arg-AMC was purchased from Bachem Bioscience Inc. (Bubendorf, Switzerland), prepared with in 15 mM CaCl₂ and concentration was determined using E₃₂₆=17,200 M⁻¹cm⁻¹ (Bunce, et al. (2011) Blood 117(1):290-298). Pooled-platelet-poor normal human plasma and FVIII deficient plasma were purchased from George King Biomedical (Overland Park, Kans.). Unless noted otherwise, all assays were done at 25° C. in assay buffer (20 mM HEPES [4-(2-hydroxyethyl)-1-piperazineethanesulfonic acid], 150 mM NaCl, 5 mM CaCl₂, 0.1% polyethylene glycol-8000, pH 7.4) and all listed reagent or protein concentrations are final concentrations for experimental conditions.

### Proteins

Plasma-derived FX, FXa, and thrombin were purified and prepared as described (Baugh, et al. (1996) J. Biol. Chem., 271:16126-16134; Buddai, et al. (2002) J. Biol. Chem., 277(29):26689-26698). Factor IXa and APC were purchased from Haemtech (Essex Junction, VT). Hirudin was purchased from Calbiochem (San Diego, Calif.). Protein concentrations were determined immediately before each experiment using the following molecular weights (Mr) and extinction coefficient (E)⁰⁻¹%: thrombin (37,500 and 1.94), FIXa (45,000 and 1.40), FX (59,000 and 1.16), FXa (46,000 and 1.16), APC (45,000 and 1.45), and PS (69,000 and 0.95), respectively (Lundblad, et al. (1976) Thrombin 1976:156-176; Di Scipio, et al. (1977) Biochemistry 16(4):698-706; Fujikawa, et al. (1974) Biochemistry 13(22):4508-4516).

### Generation of Recombinant FVIII Proteins

Baby hamster kidney (BHK) cell lines stably expressing wild-type B-domain deleted FVIII (FVIII-WT) were developed and purified (Pittman, et al. (1993) Blood 81(11):2925-2935; Sabatino, et al. (2009) Blood 114(20):4562-4565). Site-directed mutagenesis of FVIII-WT cDNA (Genescript; Piscataway, N.J.) was employed to introduce Arg to Gln mutations at FVIII APC cleavage sites, Arg336 and Arg562 (FIG. 1B). Factor VIII proteins were purified (˜3 mg each) from 24 liters of conditioned media using ion-exchange chromatography (Sabatino, et al. (2009) Blood 114(20):4562-4565). Recombinant FVIII concentrations were determined by absorbance at 280 nm based on an E^{0.1%} of 1.60 and molecular weight (Mr)=165,000 (Curtis, et al. (1994) J. Bio. Chem., 269(8):6246-6251). As a control, recombinant FVIII-R372Q was similarly generated.

### Plasma Assays

FVIII specific activity was determined by an aPTT-based-1-stage clotting assay (Siner, et al. (2016) JCI Insight., 1(16):e89371). Thrombin generation in platelet-poor plasma was determined as described with modifications (Bunce, et al. (2011) Blood 117(1):290-298). Factor VIII-deficient plasma was reconstituted with 1 nM FVIII or 0.2 nM FVIIIa with 4 μM PCPS. To generate FVIIIa, FVIII (1.5 nM) was incubated with thrombin (30 nM) for 30 seconds and quenched with hirudin (60 nM). In FVIII reconstituted plasma, thrombin generation was initiated using 1 pM or 30 pM FXIa in human and murine plasma, respectively. In FVIIIa reconstituted plasma, thrombin generation was initiated with 10 pM FXIa and 400 pM FXIa in human and mouse plasma, respectively. The concentration of FVIIIa and FXIa in these assays were chosen to generate similar peak thrombin and lag times relative to experiments with FVIII in analogous HA plasma (Table 1). The reaction was initiated with 0.5 mM Z-Gly-Gly-Arg-AMC (Bachem Bioscience Inc.) in 0.5 mM CaCl₂. Fluorescence was measured over 90 minutes at 37° C. or 33° C. for human and mouse plasma, respectively, by a Spectromax® M2 (Molecular Devices; San Jose, Calif.) with 360 nm excitation and 460 nm emission wavelengths. Raw fluorescence values were compared to a thrombin calibration curve using a thrombin calibrator (Technothrombin® thrombin generation assay calibrator set) to convert data to nM thrombin and thrombin generation curves (nM/time) and analyzed to determine peak thrombin generation and lag time. APC was used because human soluble thrombomodulin (sTm) does not cross-react with mouse APC.

TABLE 1
Peak thrombin and lag time values in plasma
reconstituted with FVIII and FVIIIa.

| Plasma | Protein | Peak Thrombin (nM) | Lag Time (min) |
|---|---|---|---|
| HA murine | FVIII-WT | 357.15 ± 6.64 | 4.25 ± 0.25 |
| | FVIII-QQ | 366.35 ± 3.96 | 4 ± 0.41 |
| | FVIIIa-WT | 234.34 ± 9.69 | 3.5 ± 0.29 |
| | FVIIIa-QQ | 155.26 ± 25.30 | 4 ± 0.0 |
| HA human | FVIII-WT | 703.31 ± 43.56 | 9.0 ± 0.4 |
| | FVIII-QQ | 678.14 ± 5.04 | 8.5 ± 0.3 |
| | FVIIIa-WT | 364.10 ± 10.19 | 6.0 ± 0.0 |
| | FVIIIa-QQ | 422.34 ± 25.25 | 5.5 ± 0.3 |
| HA/FVL | FVIII-WT | 288.51 ± 16.97 | 4.75 ± 0.85 |
| murine | FVIII-QQ | 288.27 ± 3.69 | 4.00 ± 0.58 |
| | FVIIIa-WT | 233.27 ± 1.86 | 3.25 ± 0.25 |
| | FVIIIa-QQ | 166.57 ± 36.07 | 3.00 ± 0.00 |

Means and standard error of the mean are displayed. Peak thrombin, maximum concentration of thrombin generated; Lag time, time to peak thrombin generation; HA, hemophilia A; HA/FVL, homozygous hemophilia A, factor V Leiden.

### Proteolytic Cleavage of FVIII by Western Blot Analysis

Factor VIII (1.5 μM) was incubated with thrombin (10 nM) for 20 minutes to generate FVIIIa and then quenched with hirudin (20 nM). To evaluate APC cleavage, FVIII (10 nM) was incubated with APC (6 nM), hirudin (6 nM), and PCPS (20 μM) for 30 minutes. Hirudin was added to purified system assays to quench possible trace thrombin contamination from commercially available APC. Samples were analyzed by western blot analysis. FVIII and FVIII cleavage products were detected by a primary antibody that recognizes the FVIII A2 domain (Fay, et al. (1991) J. Biol. Chem., 266(14):8957-8962) (GMA-012, Green Mountain Antibodies; Burlington, Vt.) and Dylight™ 800 secondary detection antibody (Rockland; Pottstown, Pa.).

### Factor VIII Enzyme Kinetic Studies and Measurement of A2 Stability

Kinetic analysis of FXa generation was performed by an intrinsic Xase assay, as described with modifications (Lollar, et al. (1989) Biochemistry 28(2):666-674). Activated FVIII (FVIIIa) was generated by incubating 25 nM FVIII with 100 nM thrombin for 30 seconds and thereafter quenched with hirudin (150 nM). Factor VIIIa (0.25 nM) was immediately combined with FIXa (20 nM) and variable FX concentrations (0-500 nM) in the presence of 20 μM PCPS. At various time intervals (0.25-2 minutes), aliquots of the reaction mixture were quenched in 20 mM HEPES, 150 mM NaCl, 25 mM EDTA, 0.1% polyethylene glycol-8000, pH 7.4. The amount of FXa in each quenched sample was assessed using Spectrozyme® Xa by measuring absorbance at 405 nm in SpectraMax® 190 Microplate reader (Molecular Devices) and comparing the results to a prepared FXa standard curve. Residual FVIII activity in the presence of APC or APC and PS incubation was performed as described, except FVIII proteins were incubated with 6 nM APC (Haemtech) or 6 nM APC and 100 nM PS in the presence of 20 μM PCPS and 6 nM hirudin for 0-60 minutes prior to thrombin activation. Evaluation of FVIIIa-A2 dissociation was performed as described, except variable concentrations of FVIII were activated (5-100 nM) with 100 nM thrombin and aliquots were removed at indicated times and assayed immediately for residual FVIIIa function in the intrinsic Xase assay (Lollar, et al. (1989) Biochemistry 28(2):666-674).

### Animals

HA-057BL/6 mice were used for in vivo studies (Bi, et al. (1995) Nat. Genet., 10(1):119-121). Homozygous HA-057BL/6 mice were bred with homozygous FV Leiden (FVL)-057BL/6 mice to generate homozygous HA/FVL-057BL/6 mice (Schlachterman, et al. (2005) J. Thromb. Haemost., 3(12):2730-2737; Cui, et al. (2000) Blood 96(13):4222-4226). Factor V Leiden (FV^{R506Q}) (FVL) is cleaved ˜10× slower than FV and is, therefore, APC resistant. Wild type C57BL/6 mice were purchased from Jackson Labs. Males and females ages 8-12 weeks were used for experiments. Animal studies were approved by Children's Hospital of Philadelphia Animal Care and Use Committee.

### Tail Clip Assay

Mice were anesthetized with isoflurane and the tail was pre-warmed to 37° C. Factor VIII protein and/or mAb 1609 (200 μL for a dose of 10 μg/mL) was injected by retro-orbital injection 3 minutes before transection of the tail at a 3 mm diameter (Xu, et al. (2009) J. Thromb. Haemost., 7(5):851-856). The tail was placed into a conical tube, blood was collected for 2 minutes and thereafter another 10 minutes into normal saline. The 10-minute samples were hemolyzed and the absorbance was measured at 575 nm to determine the total hemoglobin present (Sambrano, et al. (2001) Nature 413(6851):74-78). Total blood loss (μL) was determined by converting sample hemoglobin content using an established standard curve of known amounts of hemolyzed murine whole blood (Ivanciu, et al. (2011) Nat. Biotechnol., 29(11):1028-1033).

### FeCl₃ Injury Model

The ferric chloride (FeCl₃) injury was performed in HA-057BL/6 mice as described (Schlachterman, et al. (2005) J. Thromb. Haemost., 3(12):2730-2737). In brief, the carotid artery was exposed and flow was measured by a Doppler probe (Model 0.5VB; Transonic Systems; Ithaca, N.Y.) placed under the artery. Approximately 3 minutes following jugular vein FVIII protein infusion, carotid artery vessel injury was performed by placing a 2 mm² filter paper soaked in 7.5% FeCl₃ on the artery adventitial surface for 2 minutes. Thereafter the filter paper was removed, the area was washed with normal saline and blood flow was continuously monitored by Doppler flow for up to 30 minutes. Time to carotid artery vessel occlusion was defined as no measurable blood flow and is reported in experimental results.

Thrombin Generation Assay with Soluble Thrombomodulin Titration

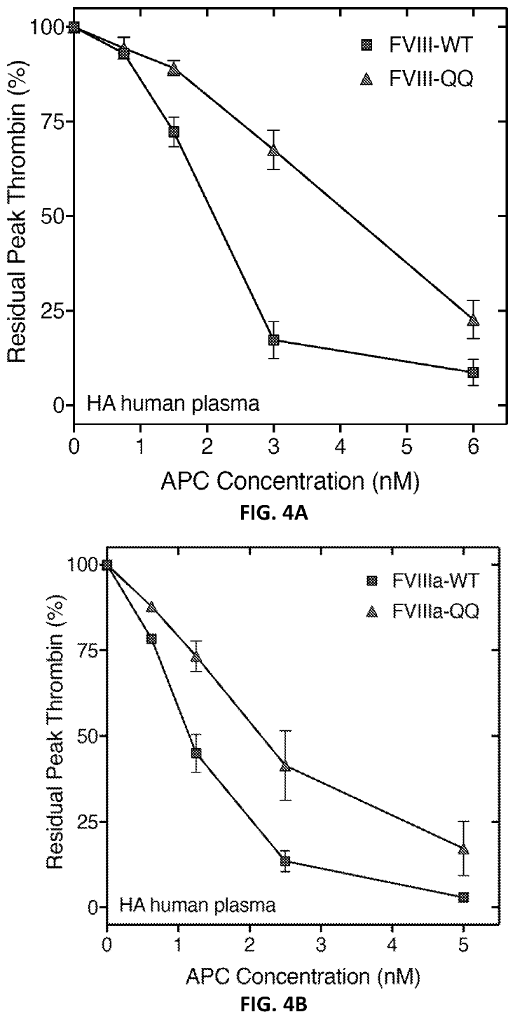

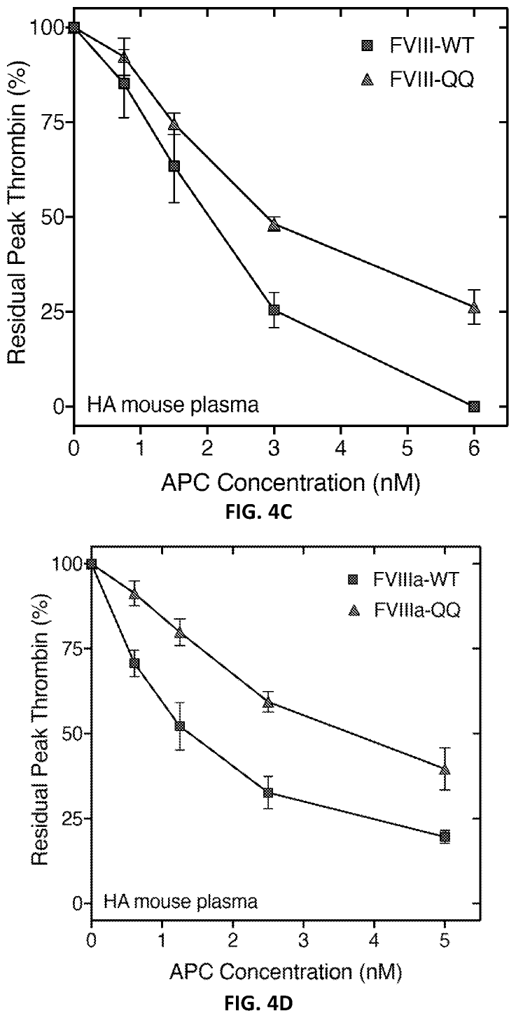

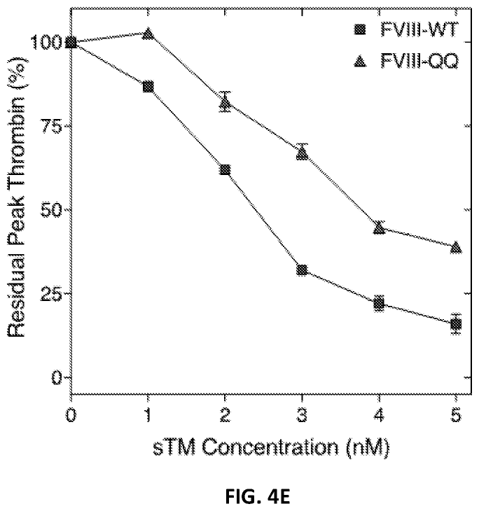

Human FVIII-deficient plasma was reconstituted with 1 nM FVIII-WT or FVIII-QQ, 4 μM PCPS, and increasing amounts of soluble thrombomodulin (sTM). Human sTM was recombinantly produced and purified as described (Bradford, et al. (2012) J. Biol. Chem., 287(36):30414-30425; Parkinson, et al. (1990) J. Biol. Chem., 265(21):12602-12610). Thrombin generation was triggered with 0.1 pM FXIa. The reaction was initiated with 0.5 mM Z-Gly-Gly-Arg-AMC (Bachem Bioscience Inc.) and 7.5 mM CaCl₂ (final concentrations). bFluorescence was measured over 90 minutes at 37° C. by a Spectromax® M2 (Molecular Devices) with 360 nm excitation and 460 nm emission wavelengths. Raw fluorescence values were compared to a thrombin calibration curve using a thrombin calibrator (Technothrombin® thrombin generation assay calibration set) to convert data to nM thrombin and thrombin generation curves (nM/time) and analyzed to determine peak thrombin generation and lag time (FIG. 4E).

### FVIII Half-Life Studies

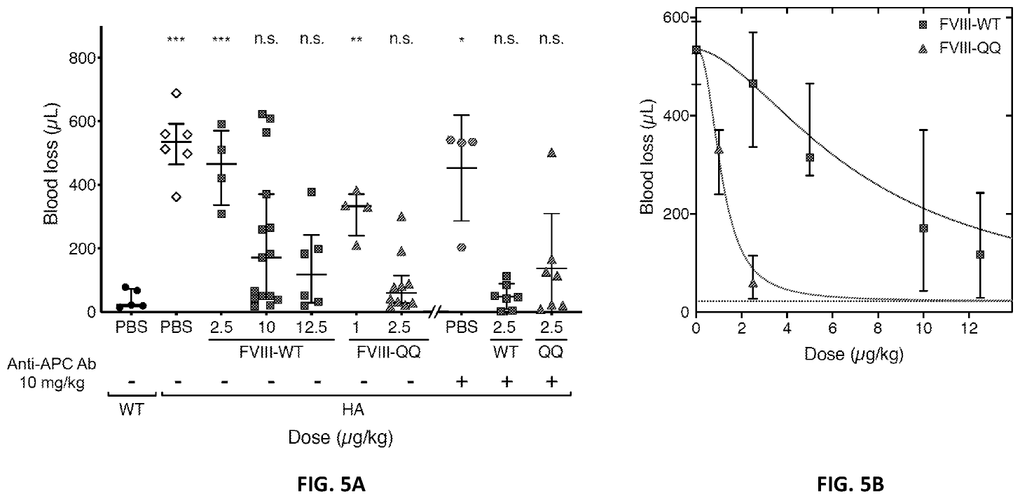

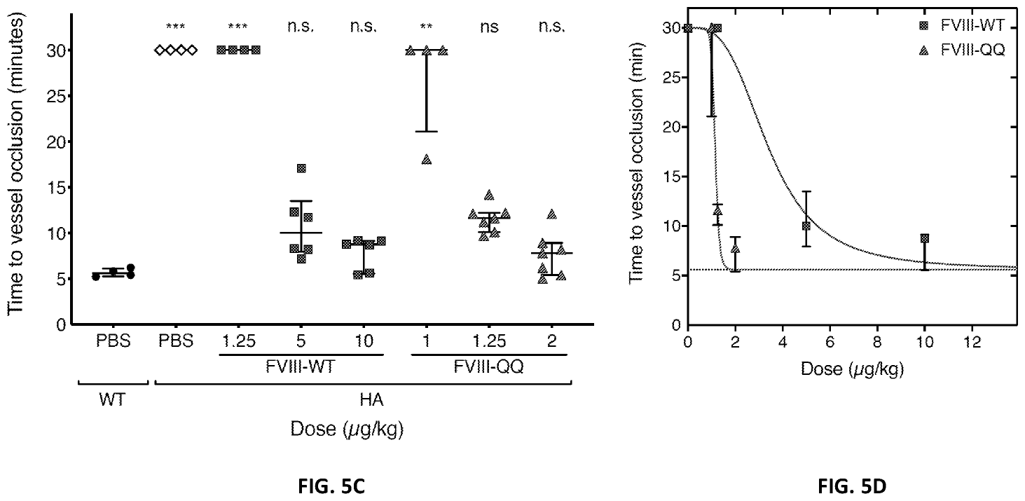

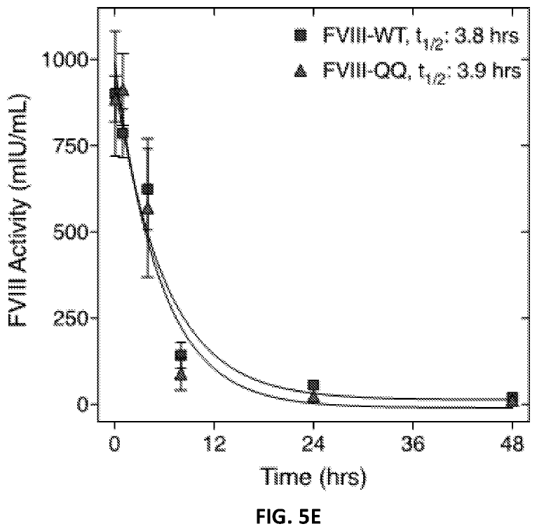

HA-057BL/6 mice were injected with 125 IU/kg of either FVIII-WT or FVIII-QQ by tail vein injection to determine FVIII half-life. Plasma samples were collected at 5 minutes, 1 hour, 4 hours, 8 hours, 24 hours, 48 hours post protein injection into 3.8% sodium citrate and snap frozen for later analysis. Factor VIII residual activity was determined using the Chromogenix Coamatic® FVIII kit (Diapharma, Louisville, Ky.) with a modified protocol decreasing the incubation times to 4 minutes (Rosen, et al. (1985) Thromb. Haemost., 54(4):818-823). Half-lives were determined by fitting the residual FVIII activity to an exponential decay curve using Prism software (Dumont, et al. (2012) Blood 119(13):3024-3030) (FIG. 5E).

### Data Analysis

Analyses were performed in Graphpad Prism 8 software. Specific statistical analysis methods are outlined in the figure legends. Steady-state kinetic parameters, K. and Vₘₐₓ, for FX activation by the intrinsic FXase were calculated by non-weighted nonlinear least-squares fits to the Michaelis-Menten equation. Results are expressed as ±standard error of the mean. Mouse injury studies were analyzed by one-way ANOVA on ranks (Kruskal-Wallis, non-parametric fit) using Dunn's Multiple Comparisons Test.

### Results

### Characterization of FVIII-WT and FVIII-QQ Procoagulant Activity

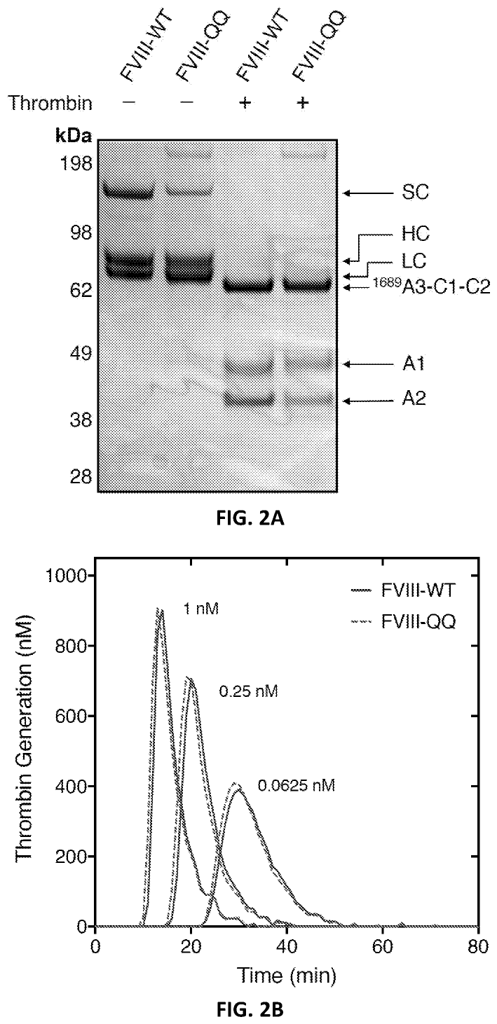

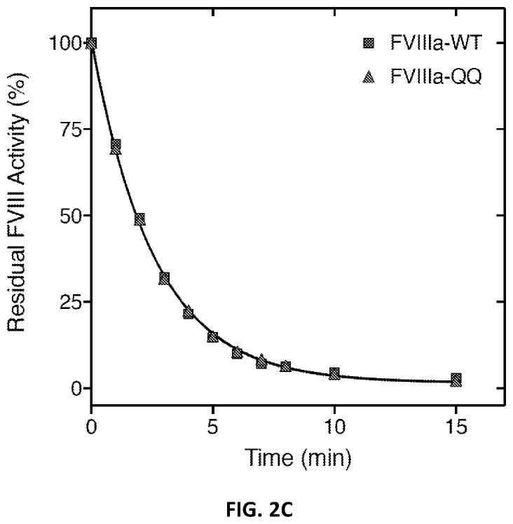

To ensure that the introduction of 2 mutations did not alter FVIII procoagulant function, FVIII-QQ was compared to FVIII-WT in different assay systems. FVIII-WT and FVIII-QQ were purified from conditioned media in their single chain (Mr=165,000) and heterodimeric forms (heavy chain, Mr=90,000 and light chain, Mr=80,000). Thrombin cleaved both proteins to yield fragments representing cleavage at R1689 (A3-C1-C2; Mr=70,000) and R740/R372 (A1, Mr=50,000 and A2, Mr=43,000), corresponding to FVIIIa (FIG. 2A).

The specific activity of FVIII-WT (9000±700 IU/mg) and FVIII-QQ (11000±900 IU/mg) were similar and consistent with commercially available B-domainless FVIII products (Table 2) (www.fda.gov/media/70399/download2014). Further, both proteins demonstrated similar peak thrombin generation, endogenous thrombin potential and lag times at varying concentrations assessed by thrombin generation assays (FIG. 2B). In a purified system, FVIIIa-WT and FVIIIa-QQ displayed similar Km and Vmax values for FX activation (Table 2) that were consistent with published values (Lollar, et al. (1994) J. Clin. Invest., 93(6):2497-2504). Importantly, the introduction of the two mutations did not affect A2-domain stability as both proteins spontaneously lost nearly-all FVIIIa activity within 15 minutes, attributed to A2-domain dissociation (FIG. 2C).

TABLE 2
Biochemical characterization of FVIII-QQ.

| | Specific Activity | Kₘ | Vₘₐₓ |
|---|---|---|---|
| | (IU/mL) | (nM) | (nM FXa/min) |
| FVIII-WT | 9000 ± 700 | 160 ± 20 | 18 ± 4 |
| FVIII-QQ | 11000 ± 900 | 201 ± 7 | 23 ± 3 |

Data are represented as means ± SEM from at least two independent experiments. Kinetic values were determined for FX activation by an intrinsic Xase assay using 0.25 nM FVIIIa, 20 nM FIXa and 0-500 nM FX in the presence of 20 μM phospholipids.

### FVIII/FVIIIa-QQ is Resistant to APC Cleavage

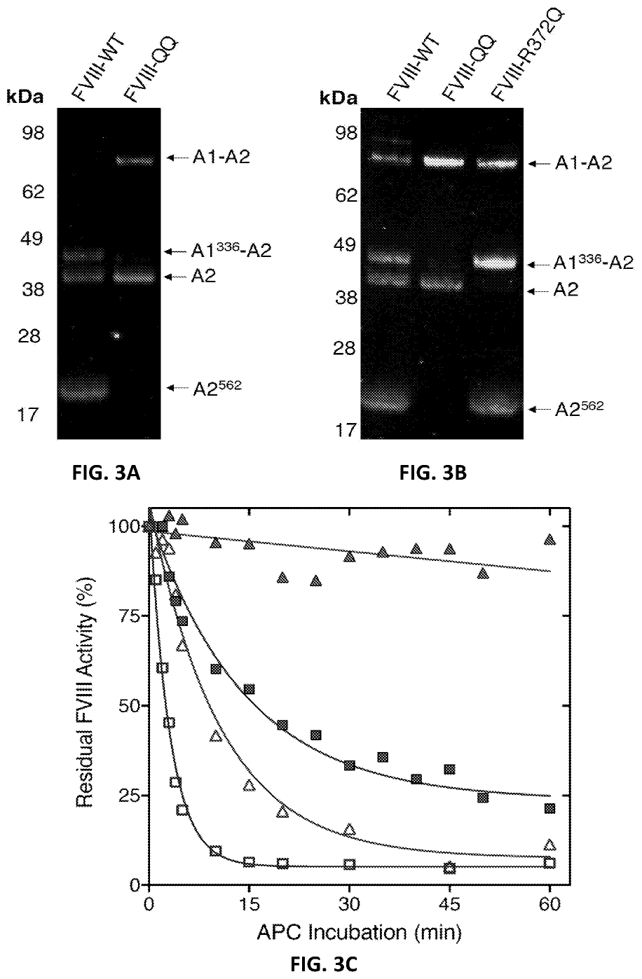

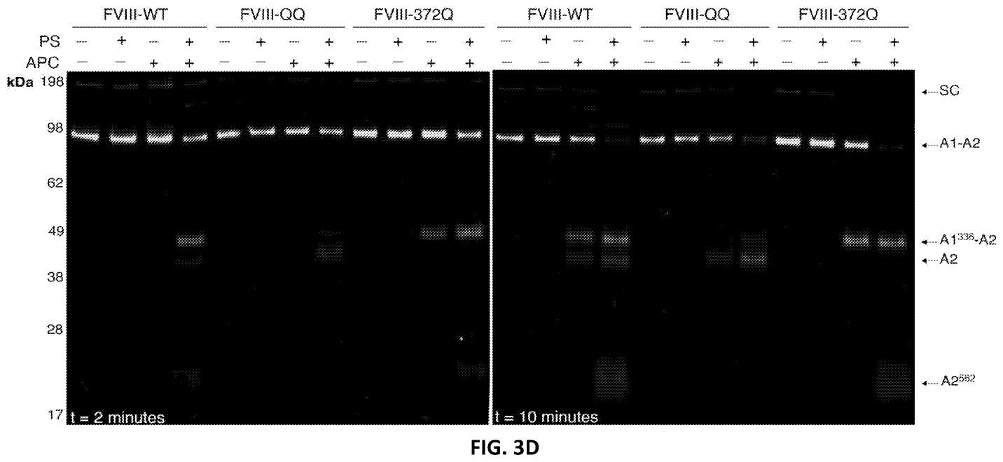

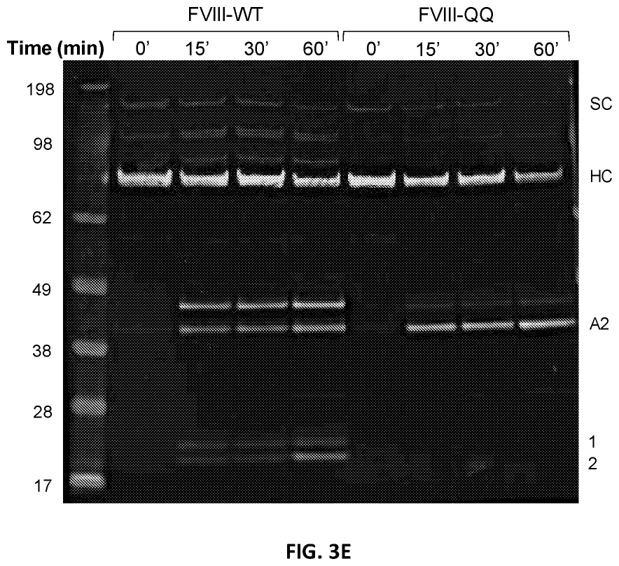

To confirm FVIII-QQ resistance to APC cleavage, FVIII-QQ and FVIII-WT were incubated with APC for 30 minutes and reaction products were evaluated by western blot analysis. As expected, APC cleavage of FVIII-WT yielded fragments consistent with cleavage at both R336 (A1³³⁶-A2) and R562 (A2⁵⁶²) while no analogous FVIII-QQ cleavage fragments were detected (FIG. 3A). Under the conditions employed, both FVIII-WT and FVIII-QQ were cleaved by APC in the A2 domain yielding a fragment consistent with cleavage at R372 (FIG. 3A). This was confirmed incubating a FVIII-R372Q mutant with APC, which did not produce the A2 fragment (FIG. 3B) and is consistent with reports of APC cleavage at FVIII thrombin cleavage sites (Fay, et al. (1991) J. Biol. Chem., 266(30):20139-20145). Further, the cleavage was APC-specific as hirudin was added to the reaction to inhibit potential trace thrombin. Consistent with FVIII-QQ being APC resistant, the protein maintained >90% activity following a 1-hour APC incubation (FIG. 3C). In contrast, FVIII-WT lost approximately 75% activity after a 1-hour APC incubation (FIG. 3C). The loss of FVIII-WT activity due to APC cleavage was confirmed by western blot analysis. FIG. 3E shows an APC incubation timecourse. WT and FVIII-QQ (450 nM) were reacted with 20 μM PCPS and 90 nM APC for a time course of 60 minutes. Western blot was visualized using A2-specific antibody (GMA-8028). Collectively, the data show that introduction of Arg336Gln and Arg562Gln mutations to FVIII blocked cleavage at these sites and conferred functional APC resistance without significant effects on other aspects of FVIII/FVIIIa procoagulant function.

Consistent with published data (Lu et al. (1996) Blood 87:4708-4717), FVIII-WT lost nearly all function within 15 minutes of combined APC and PS incubation (FIG. 3C). Surprisingly, combined PS and APC incubation also accelerated FVIII-QQ loss of function, albeit to a lesser extent than FVIII-WT, indicating a role of APC/PS-mediated inactivation outside of the R336 and R562 cleavage sites. Western blotting revealed that both FVIII-WT and FVIII-QQ demonstrated enhanced APC cleavage at R372 in the presence of PS (FIG. 3D). Thrombin cleavage at R372 converts the FVIII heterodimer to the FVIIIa heterotrimer. PS cofactor function may accelerate APC cleavage at R336 and R562 as well as R372 resulting in heterotrimer formation indicating measured loss of FVIII function in this in vitro system following APC and PS incubation likely reflects APC cleavage at R336 and R562 as well as spontaneous A2-domain dissociation after APC cleavage at R372. The physiologic significance of APC cleavage at R372 cleavage is unclear given R372 is already cleaved following thrombin-mediated FVIIIa heterotrimer formation.

To determine the impact of APC on FVIIIa inactivation in plasma, human HA plasma was reconstituted with physiologic amounts of FVIII (1 nM) and APC. Factor VIII procoagulant activity was assessed by a thrombin generation assay. In the presence of increasing APC concentrations, FVIII-QQ demonstrated greater thrombin generation, as assessed by peak thrombin, relative to FVIII-WT. Factor VIII-QQ reconstituted HA plasma lost approximately 30% activity while FVIII-WT reconstituted HA plasma lost 80% activity in the presence of 3 nM APC (FIG. 4A). Comparable results were observed with increasing sTM concentrations in place of APC (FIG. 4E). In this assay system, FVIIIa and FV/FVa are inactivated by APC, which likely explains why thrombin generation decreases using FVIII-QQ. Nonetheless, reconstituted plasma with FVIII-QQ, when compared to FVIII-WT, is resistant to APC. Similar thrombin generation studies were conducted using FVIIIa. Here, FVIII-QQ and FVIII-WT were rapidly activated by thrombin and then added to human HA plasma. As was observed with the procofactor, FVIIIa-QQ demonstrated greater thrombin generation in the range of APC concentrations tested relative to FVIIIa-WT (FIG. 4B). Since FVIIIa is added to the system prior to initiating thrombin generation, A2-dissociation likely plays a major role in FVIIIa regulation in this experimental system. However, even with enhanced A2-dissociation conditions, differences in APC sensitivity between FVIIIa-WT and FVIIIa-QQ are observed. Similar results were observed using HA murine plasma reconstituted with either FVIII (FIG. 4C) or FVIIIa (FIG. 4D). The more marked decrease in thrombin generation of FVIII/FVIIIa-WT relative to FVIII/FVIIIa-QQ in the presence of APC supports the role of APC in FVIIIa inactivation in this HA plasma-based system.

### APC-Resistant FVIII Improves Hemostatic Efficacy in HA Mouse Injury Models

The tail clip and FeCl₃ assays were performed on HA mice to evaluate the relative effect of FVIII-WT versus FVIII-QQ in vivo. The tail clip assay demonstrated a dose-dependent decrease in blood loss for both FVIII-QQ and FVIII-WT (FIG. 5A). The dose of FVIII-QQ that normalized blood loss (2.5 μg/kg) was lower than the dose of FVIII-WT that normalized blood loss (10 μg/kg), which is consistent with an in vivo contribution of APC in FVIIIa regulation. To ensure the observations were APC-specific, the tail clip assay was repeated in the presence of an antibody that inhibits mouse APC anticoagulant function, mAb1609 (FIG. 5A) (Xu, et al. (2009) J. Thromb. Haemostasis 7(5):851-856). Infusion of mAb1609 in HA mice did not confer a hemostatic effect by itself and blood loss was similar to PBS controls. However, administration of mAb1609 with 2.5 μg/kg FVIII-WT (the dose of FVIII-QQ that normalized blood loss) in HA mice reduced blood loss consistent with hemostatically normal controls. Unlike FVIII-WT, FVIII-QQ blood loss was the same with or without mAb1609. These results indicate that the superior hemostatic efficiency of FVIII-QQ in vivo is specific to its resistance to APC cleavage.

Based on recovery studies (FIG. 5E), the dose of FVIII-WT needed to normalize blood loss approximated a plasma FVIII activity of 67% of normal and consistent with publications (Nguyen, et al. (2017) J. Thromb. Haemost., 15(1):110-121; Siner, et al. (2013) Blood 121(21):4396-4403). Quantitatively, the EC50 of FVIII-QQ was 6-7 fold lower than FVIII-WT (1.1 μg/kg and 7.4 μg/kg, respectively) while the EC80 was 8-9 fold lower than FVIII-WT (2.2 μg/kg and 18.6 μg/kg, respectively) (FIG. 5B, Table 3). Analogous to the tail clip assay, the dose of FVIII-QQ (2 μg/kg) that normalized time to vessel occlusion in the FeCl₃ assay was lower than the dose of FVIII-WT (10 μg/kg). In the FeCl₃ assay, the EC50 of FVIII-QQ was 3-fold lower than FVIII-WT (1.2 μg/kg and 3.4 μg/kg, respectively), while the EC₈₀ of FVIII-QQ was 8-fold lower than FVIII-WT (1.5 μg/kg and 12.1 μg/kg, respectively) (FIG. 5D, Table 3). The half-lives and recovery of FVIII-WT and FVIII-QQ were similar in HA mice (FIG. 5E). These data demonstrate that in large vessel injury models, APC has a critical role in the in vivo regulation of FVIIIa such that resistance to APC-cleavage confers a hemostatic benefit.

TABLE 3
In vivo hemostatic function of FVIII-QQ relative to FVIII-WT.

| | Tail Clip | Tail Clip | FeCl₃ | FeCl₃ |
|---|---|---|---|---|
| | EC₅₀ | EC₈₀ | EC₅₀ | EC₈₀ |
| | (μg/kg) | (μg/kg) | (μg/kg) | (μg/kg) |
| FVIII-WT | 7.6 | 16.5 | 3.4 | 12.1 |
| FVIII-QQ | 1.1 | 2.0 | 1.2 | 1.5 |
| FVIII-QQ fold | 6.9 | 8.3 | 2.8 | 8.1 |
| improvement | | | | |

EC_{50/80}, Dose of FVIII required for 50% or 80% of normal blood loss or normal time to vessel occlusion; FeCl₃, 7.5% ferric chloride injury model.

### Effect of APC on FVIII/FVIIIa Function in HA/FVL Mouse Plasma and Injury Models

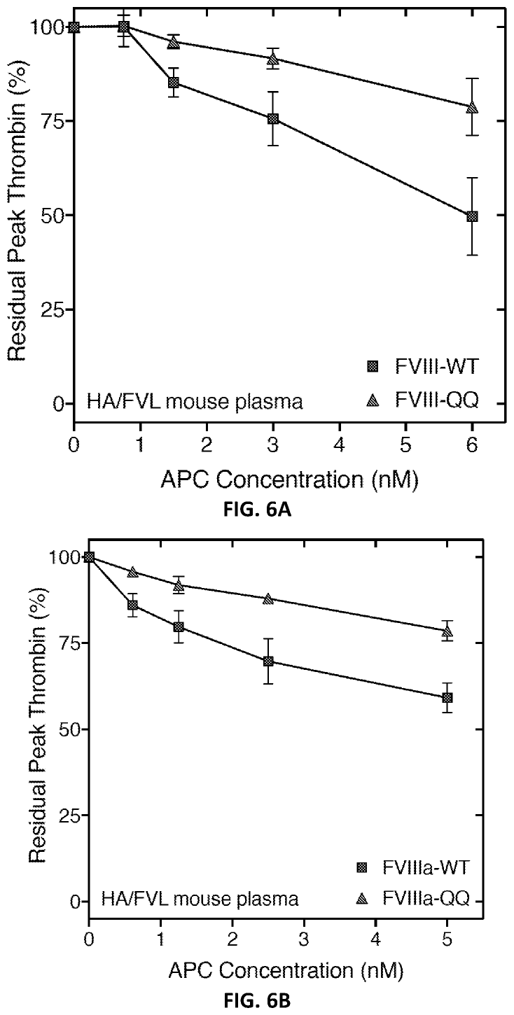

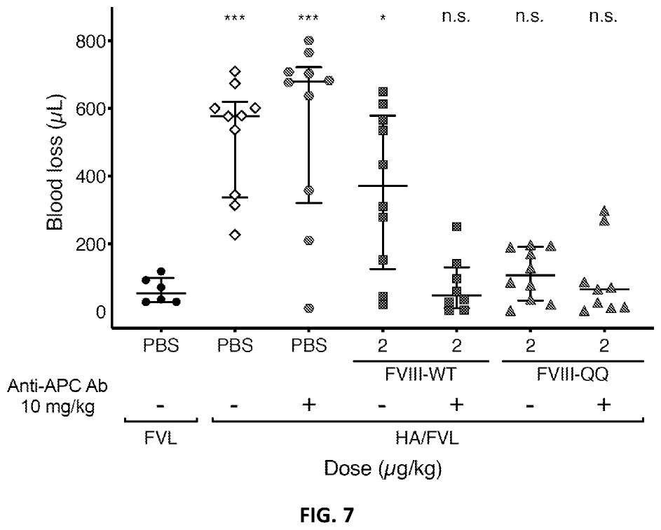

To further isolate the contribution of APC cleavage to FVIIIa inactivation, homozygous HA/FVL mice were generated. Studies of HA/FVL mice found that FVL confers a modest improvement in microvascular bleeding with no observable effect in large vessel injury models (Schlachterman, et al. (2005) J. Thromb. Haemost., 3(12):2730-2737). First, thrombin generation assays were repeated in mouse HA/FVL plasma reconstituted with FVIII-WT or FVIII-QQ in the presence of varying concentrations of APC. As expected, inactivation of FVIII-WT and FVIII-QQ in HA plasma was markedly different compared to HA/FVL plasma (compare FIG. 4C versus FIG. 6A). However, in HA/FVL plasma, residual peak thrombin values were still higher with FVIII-QQ relative to FVIII-WT for all APC concentrations (FIG. 6A). Similar results were obtained when plasma was reconstituted with FVIIIa-WT versus FVIIIa-QQ (FIG. 6B). Next, the tail clip assay was repeated in HA/FVL mice comparing the hemostatic effect of FVIII-QQ relative to FVIII-WT. While administration of FVIII-WT (2 μg/kg) in HA/FVL mice had a modest effect on blood loss, FVIII-QQ (2 μg/kg) normalized blood loss to hemostatically normal controls (FIG. 7). These data, using a system in which FV inactivation is severely blunted, show that the inactivation of FVIIIa by APC must play an important role in regulating clot formation in vivo.

To further document this, the tail clip assay using HA/FVL mice was repeated in the presence of an antibody that inhibits mouse APC anticoagulant function, mAb1609 (Xu, et al. (2009) J. Thromb. Haemost., 7(5):851-856). As expected, infusion of mAb1609 in HA/FVL mice did not confer a hemostatic effect by itself and blood loss was similar to PBS controls (FIG. 7). These data, using a system in which FV is resistant to APC inactivation, show that the inactivation of FVIIIa by APC must play an important role in regulating clot formation in vivo. Like observations in HA mice, infusion of PBS and mAb1609 into HA/FVL mice resulted in similar blood loss as HA/FVL PBS controls (FIG. 6). Analogous to observations in HA mice, administration of mAb1609 with FVIII-QQ (2.5 μg/kg) in HA/FVL mice did not appreciably alter blood loss while administration with FVIII-WT (2.5 μg/kg) reduced blood loss to levels seen in hemostatically normal controls. Thus, eliminating APC anticoagulant function (mAb1609) or removing APC procoagulant substrates (FV-Leiden and FVIII-QQ) effectively resulted in a similar pro-hemostatic effect. These results indicate that the superior hemostatic efficiency of FVIII-QQ in vivo is specific to its resistance to APC cleavage.

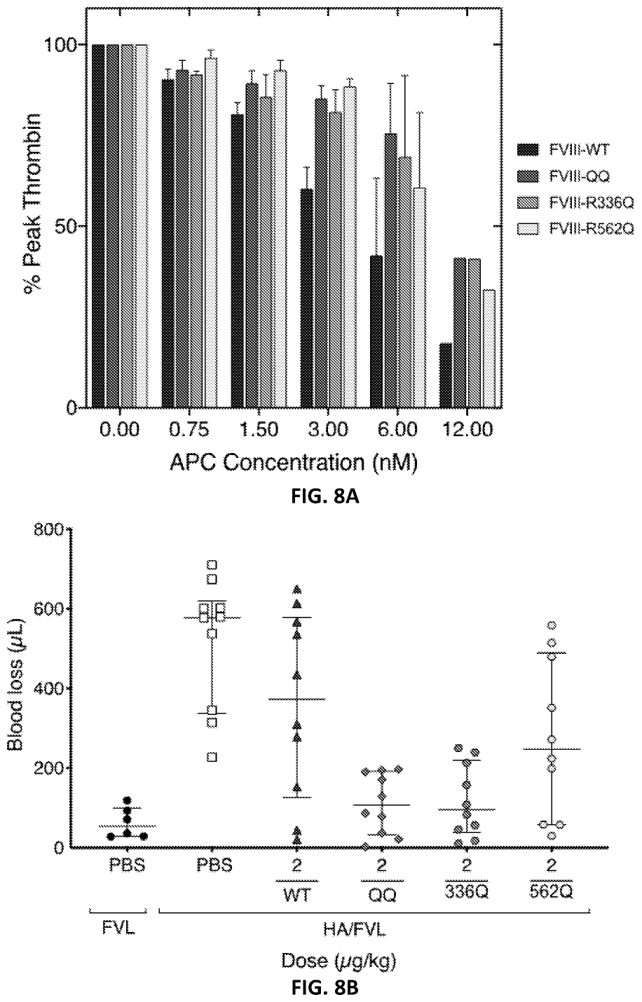

Lastly, single mutants of FVIII were evaluated in a HA/FVL plasma thrombin generation assay with increasing concentrations of APC. As seen in FIG. 8A, FVIII-R336Q and FVIII-R562Q retained superior activity compared to WT. The single mutants were also tested in a hemostatic injury model in the HA/FVL mice. As seen in FIG. 8B, FVIII-R336Q and FVIII-R562Q were superior to WT FVIII.

The studies presented herein with FVIII-QQ show that APC has an unexpectedly substantial role in FVIIIa regulation in vivo. FVIII-QQ demonstrated APC resistance without altering procoagulant function or A2-domain stability relative to FVIII-WT. Simultaneously, FVIII-QQ's resistance to APC cleavage conferred improved hemostatic function in HA mice relative to FVIII-WT in large vessel injury models. The advantage of FVIII-QQ over FVIII-WT was abrogated by an APC inhibitory antibody, confirming the enhanced hemostatic efficacy of FVIII-QQ was APC specific. These data demonstrate the in vivo significance of APC cleavage in FVIIIa inactivation.

There are two known mechanisms of FVIIIa inactivation. Biochemical studies suggest that spontaneous A2-domain dissociation is predominantly responsible for FVIIIa inactivation and the contribution by APC is relatively insignificant based on rates of inactivation (Lollar, et al. (1991) J. Biol. Chem., 266(19):12481-12486; Fay, et al. (1991) J. Biol. Chem., 266(14):8957-8962; Lollar, et al. (1992) J. Biol. Chem., 267(33):23652-23657). Indeed, data shows a rapid, spontaneous, A2-domain dissociation and comparably slow APC-mediated cleavage, with the resultant FVIIIa inactivation taking place over minutes versus hours, respectively (Lollar, et al. (1991) J. Biol. Chem., 266(19):12481-12486; Lollar, et al. (1992) J. Biol. Chem., 267(33):23652-23657; Fay, et al. (1991) J. Biol. Chem., 266(30):20139-20145). As such, the sensitivity of FVIII/FVIIIa-WT to increasing APC concentrations in plasma and enhanced hemostatic effect of FVIII-QQ in mouse injury models are surprising in the context of biochemical data of FVIIIa regulation. While the data does not exclude A2-domain dissociation as an important mechanism of FVIIIa regulation, they indicate that—in complete contrast to in vitro rate constant predictions—A2-dissociation is not the sole relevant mechanism of in vivo FVIIIa regulation.

Notably, interactions within the intrinsic Xase complex that alter A2-domain dissociation kinetics that are difficult to simultaneously model and may result in discordance between determined in vitro rates of FVIIIa inactivation and observed in vivo hemostatic effect. This underscores the importance of pairing in vitro analysis with in vivo investigation to ascertain the impact of a particular regulatory mechanism. For example, the binding affinity of the A2-domain within the FVIIIa heterotrimer is nearly 300-fold higher than plasma FVIII concentrations, which would suggest rapid A2-domain dissociation occurs in vivo when FVIIIa is free (Lollar, et al. (1992) J. Biol. Chem., 267(33):23652-23657; Parker, et al. (2006) J. Biol. Chem., 281(20):13922-13930). However, the concentration of FVIIIa at the site of injury is unknown and it is not clear how much of it is bound to various ligands versus actually free. Importantly, FIXa is well known to stabilize the A2-domain within the FVIIIa heterotrimer in the intrinsic Xase complex (Fay, et al. (1996) J. Biol. Chem., 271(11):6027-6032; Lollar, et al. (1984) Blood 63(6):1303-1308). Further, APC cleavage alters the orientation of the A2-domain, reducing FVIIIa affinity for both FIXa and FX (Regan, et al. (1996) J. Biol. Chem., 271(8):3982-3987; Rosenblum, et al. (2002) J. Biol. Chem., 277(14):11664-11669). Thus, it is unclear if the A2-domain reaches equilibrium within the FVIIIa heterotrimer while assembled within the intrinsic Xase enzyme complex at the site of injury. Additionally, both protein S (PS), and FV (both absent in the direct measurements of FXa generation), are reported to be synergistic cofactors of APC-mediated cleavage of FVIIIa (Dahlback, et al. (1994) Proc. Natl. Acad. Sci., 91:1396-1400; Shen, et al. (1994) J. Biol. Chem., 269:18735-18738; Fay, et al. (1991) J. Biol. Chem., 266(30):20139-20145; Lu, et al. (1996) Blood 87(11):4708-4717). The present studies in reconstituted HA or HA/FVL plasma with FVIIIa and in vivo injury models permitted analysis of FVIIIa function with concurrent mechanisms of FVIIIa regulation (APC-mediated proteolysis and A2-domain dissociation) in the presence of PS and FV. With this comprehensive assessment, the importance of APC regulation of FVIIIa in vivo is shown.

In addition to APC, FIXa and FXa have demonstrated ability to cleave FVIIIa residues 336 and 562, respectively (Eaton, et al. (1986) Biochemistry 25(2):505-512; Lamphear, et al. (1992) Blood 80(12):3120-3126; Nogami, et al. (2003) J. Biol. Chem., 278(3):1634-1641). By disrupting these cleavage sites in the FVIII-QQ mutant, the potential role of FIXa and FXa-mediated FVIIIa cleavage in regulation of the intrinsic Xase complex was also eliminated (Nogami, et al. (2003) J. Biol. Chem., 278(3):1634-1641; Regan, et al. (1996) J. Biol. Chem., 271(8):3982-3987).

The use of a gain-of-function FVIII transgene for HA gene transfer may overcome vector dose-dependent safety and efficacy limitations, decrease vector manufacturing demands and improve efficacy (George, L. A. (2017) Hematology 2017(1):587-594). This approach has been successfully adapted for hemophilia B gene therapy efforts such that all currently enrolling clinical trials now use a high-specific-activity FIX variant, FIX-Padua (George, et al. (2017) New Eng. J. Med., 377(23):2215-2227; Chowdary, et al. (2020) Res. Pract. Thromb. Haemost., 4(Suppl 1); Majowicz, et al. (2020) Haemophilia 26(S4):20; Simioni, et al. (2009) New Eng. J. Med., 361(17):1671-1675). Further, the first successful HA gene therapy trial observed an unexpected decline in FVIII expression (Rangarajan, et al. (2017) New Eng. J. Med., 377(26):2519-2530; Pasi, et al. (2020) New Eng. J. Med., 382(1):29-40). One proposed etiology is that FVIII expression induces an unfolded protein response and endoplasmic reticulum stress, which has been demonstrated in mammalian cell culture and liver-directed gene transfer in mice, resulting in loss of expression (Malhotra, et al. (2008) Proc. Natl. Acad. Sci., 105(47):18525-18530; Lange, et al. (2016) Mol. Ther. Meth. Clin. Dev., 3:16064; Poothong, et al. (2020) Blood 135(21):1899-1911; Becker, et al. (2004) Thromb. Haemost., 92(1):23-35; Brown, et al. (2011) J. Biol. Chem., 286(27):24451-24457). This supports use of a gain of function FVIII variant to impart similar, but durable, efficacy at lower levels of transgene expression. The FVIII-QQ doses required to normalize blood loss and clot formation in injury models were consistently approximately 5-fold lower than FVIII-WT. The enhanced hemostatic function of FVIII-QQ relative to FVIII-WT is higher than previously described gain-of-function FVIII variants (Pipe, et al. (1997) Proc. Natl. Acad. Sci., 94:11851-11856; Zakas, et al. (2017) Nat. Biotech., 35(1):35-37; Leong, et al. (2015) Blood 125(2):392-398; Wakabayashi, et al. (2008) Blood 112(7):2761-2769). In summary, the data demonstrate APC has a critical role in the in vivo regulation of FVIIIa, which was exploited to develop novel hemophilia therapeutics.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A Factor VIII (FVIII) variant comprising a substitution mutation of the Arg at position 336 and/or the Arg at position 562.

2. The FVIII variant of claim 1, wherein the Arg at position 336 and/or the Arg at position 562 is substituted with Gln.

3. The FVIII variant of claim 1, wherein the Arg at position 336 is substituted with Gln.

4. The FVIII variant of claim 1, wherein the Arg at position 562 is substituted with Gln.

5. The FVIII variant of claim 1, wherein the Arg at position 336 is substituted with Gln and the Arg at position 562 is substituted with Gln.

6. The FVIII variant of claim 1, wherein the variant lacks the B domain or the B domain has been replaced by a peptide linker.

7. The FVIII variant of claim 1, wherein said FVIII comprises amino acids 1-740 and 1649-2332 of SEQ ID NO: 1.

8. The FVIII variant of claim 1, wherein said FVIII comprises amino acids 1-740 and 1690-2332 of SEQ ID NO: 1.

9. A composition comprising at least one FVIII variant of any one of claims 1-8 and at least one pharmaceutically acceptable carrier.

10. A method for treatment of a hemostasis related disorder in a patient in need thereof comprising administration of a therapeutically effective amount of the FVIII variant of any one of claims 1-8 in a pharmaceutically acceptable carrier.

11. The method of claim 10, wherein said hemostasis related disorder is hemophilia.

12. An isolated nucleic acid molecule encoding the FVIII variant of any one of claims 1-8.

13. The nucleic acid molecule of claim 12, wherein said FVIII variant comprises a signal peptide.

14. An expression vector comprising the nucleic acid molecule of claim 12 operably linked to a regulatory sequence.

15. The vector of claim 14, selected from the group consisting of an adenoviral vector, an adenovirus-associated vector, a retroviral vector, a plasmid, and a lentiviral vector.

16. A host cell comprising the vector of claim 15.

17. The host cell of claim 16, wherein said host cells are human cells.

18. A method for treatment of a hemostasis related disorder in a patient in need thereof comprising administration of a therapeutically effective amount of the vector of claim 14 in a pharmaceutically acceptable carrier.

19. The method of claim 18, wherein said hemostasis related disorder is hemophilia.

20. The activated form of the FVIII variant of any one of claims 1-8.

21. A method for reducing blood loss in a patient in need thereof comprising administration of a therapeutically effective amount of the FVIII variant of any one of claims 1-8 in a pharmaceutically acceptable carrier.


SEQUENCE LISTING (3 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 19 aa, Artificial Sequence
  signal peptide
MQIELSTCFFLCLLRFCFS

SEQ ID NO: 3 - protein, 14 aa, Artificial Sequence
  peptide linker
SFSQNPPVLKRHQR